(19) United States
(12) Patent Application Publication
(10) Pub. No.: US 2025/0326704 A1
(43) Pub. Date: Oct. 23, 2025

(54) 1,3-BUTYLENE GLYCOL COMPOSITIONS AND METHODS OF USE

(71) Applicant: Genomatica, Inc., San Diego, CA (US)

(72) Inventors: Fasil A. Tadesse, San Diego, CA (US); Aakash Parikh, San Diego, CA (US)

(21) Appl. No.: 18/853,360

(22) PCT Filed: Apr. 10, 2023
(86) PCT No.: PCT/US2023/065599

§ 371 Date: Oct. 1, 2024

Related U.S. Application Data
(60) Provisional application No. 63/329,555, filed on Apr. 11, 2022.

(51) Int. Cl. C07C 31/20

(52) U.S. Cl. CPC C07C 31/207

(57) ABSTRACT

Provided herein are methods, compositions, and uses for a bioderived 1,3 butylene glycol (1,3-BG) characterized by one or more odor-active regions on a chromatograph. Various methods of producing and characterizing an odorless and a fragrant bioderived 1,3-BG for cosmetic uses are also provided.

### RELATED APPLICATIONS

This application is a U.S. National Stage Application under 35 U.S.C. § 371 of International Patent Application No. PCT/US2023/065599, filed Apr. 10, 2023, which claims priority to U.S. Provisional Application Ser. No. 63/329,555, filed on Apr. 11, 2022; disclosures of each of which are hereby incorporated by reference in their entireties.

### BACKGROUND OF THE INVENTION

1,3-butylene glycol (1,3-BG) is a commonly used organic solvent for food flavoring and cosmetic compositions. 1,3-BG is a four carbon diol traditionally produced in a chemical process from petroleum derived acetylene via its hydration (“petro-BG”). 1,3-BG produced using petroleum sources is costly, lacks sustainability, has irritant properties, and does not meet user preferences for cosmetic use. Therefore, new 1,3-BG formulations and methods of generating, characterizing, and using 1,3-BG are needed to improve sustainability, meet quality standards, and align with consumer preferences.

### SUMMARY OF THE INVENTION

Provided herein are bioderived 1,3-butylene glycol (1,3-BG) compositions, wherein the compositions comprise: an odor-active region having a relative acquisition time of 1.328, wherein in GC-O analysis under specified conditions the relative acquisition time of 1,3-BG is 1.0. Further provided herein are compositions, wherein the odor-active region having a relative acquisition time of 1.328 comprises an odor selected from roast and/or exhaust. Further provided herein are compositions, wherein the compositions further comprise (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, or (d) a solventy odor-active region having a relative acquisition time of 0.711, wherein in GC-O analysis under specified conditions the relative acquisition time of 1,3-BG is 1.0. Further provided herein are compositions, wherein the odor in the odor-active region having a relative acquisition time of 1.328 is below the perception threshold. Further provided herein are compositions, wherein (a) the fruity and/or solventy odor-active region having a relative acquisition time of 0.594 is below the perception threshold; (b) the fruity odor-active region having a relative acquisition time of 0.628 is below the perception threshold; (c) the citrus and/or peel odor-active region having a relative acquisition time of 0.655 is below the perception threshold, or (d) the solventy odor-active region having a relative acquisition time of 0.711 is below the perception threshold.

Provided herein are bioderived 1,3-butylene glycol (1,3-BG) compositions, wherein the compositions comprise: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, or (d) a solventy odor-active region having a relative acquisition time of 0.711, wherein in GC-O analysis under specified conditions the relative acquisition time of 1,3-BG is 1.0. Further provided herein are compositions, wherein the compositions further comprise an odor-active region having a relative acquisition time of 1.328, wherein in GC-O analysis under specified conditions the relative acquisition time of 1,3-BG is 1.0. Further provided herein are compositions, wherein the odor-active region having a relative acquisition time of 1.328 comprises an odor selected from roasty and/or exhaust. Further provided herein are compositions, wherein (a) the fruity and/or solventy odor-active region having a relative acquisition time of 0.594 is below the perception threshold; (b) the fruity odor-active region having a relative acquisition time of 0.628 is below the perception threshold; (c) the citrus and/or peel odor-active region having a relative acquisition time of 0.655 is below the perception threshold, or (d) the solventy odor-active region having a relative acquisition time of 0.711 is below the perception threshold. Further provided herein are compositions, wherein the odor in the odor-active region having a relative acquisition time of 1.328 is below the perception threshold.

Provided herein are bioderived 1,3-butylene glycol (1,3-BG) compositions, wherein the compositions comprise: an odor concentration at or lower than the perception threshold at one or more odor-active regions having a relative acquisition time selected from the group consisting of 0.544, 0.583, 0.589, 0.594, 0.60, 0.605, 0.616, 0.622, 0.628, 0.633, 0.655, 0.678, 0.683, 0.689, 0.694, 0.70, 0.705, 0.711, 0.739, 0.744, 0.750, 0.755, 0.761, 0.767, 0.80, 0.816, 0.817, 0.833, 0.872, 0.928, 0.972, 0.983, 1.017, 1.033, 1.117, 1.139, 1.211 and 1.405, wherein in GC-O analysis under specified conditions the relative acquisition time of 1,3-BG is 1.0. Further provided herein are compositions, wherein the one or more odor-active regions contain an odor selected from the group consisting of fruity, sweet, green, pungent, cheesy, solventy, mushroom, metallic, brothy, waxy, earthy, vegetal, corn chip, plastic, grassy, floral, citrus, peel, sulfuric, floral (rosy), acrylic, pungent, grape wine, medicinal, roasty, nutty, bready, cooked potato, pyrazine-like, plastic-like, dough, cooked rice, stinky, green floral, burnt plastic, cooked pasta, fried, acid, potato, pickled plum, green bell pepper, celery, exhaust, lactone-like and estery. Further provided herein are compositions, wherein the compositions further comprise an odor concentration at or lower than the perception threshold at an odor-active region having a relative acquisition time of 1.328 and having an odor selected from roasty and/or exhaust. Further provided herein are compositions, wherein the compositions further comprise an odor concentration at or lower than the perception threshold at an odor-active region having a relative acquisition time of 0.628 and having a fruity odor. Further provided herein are compositions, wherein the one or more odor-active regions comprises a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30 31, 32, 33, 34, 35, 36 or 37 odor-active regions. Further provided herein are compositions, wherein the odor concentration at the one or more odor-active regions is lower than the perception threshold. Further provided herein are compositions, wherein the odor concentration at the one or more odor-active regions is at least 50% lower than the perception threshold. Further provided herein are compositions, wherein the odor concentration at the one or more odor-active regions is between 55% and 100% lower than the perception threshold.

Provided herein are compositions, wherein the compositions comprise: the bioderived 1,3-butylene glycol (1,3-BG or 1,3-bioBG) provided herein; and a product ingredient, wherein the product ingredient is a cosmetic, fragrance, a personal care product, a flavoring substance, a food additive or a drink additive. Further provided herein are compositions, wherein the bioderived 1,3-BG is included as a humectant, a solvent, a viscosity controlling agent, a diluent, a fragrance enhancer, or a flavor enhancer.

### BRIEF DESCRIPTION OF THE DRAWINGS

FIG. 1 shows a block flow diagram for deodorization of bioderived 1,3-BG using a caustic reaction followed by multi-step distillation. Values were normalized to 100 kg of feed. Acronym Key: WFE: wiped film evaporator; T: temperature; SPE: solid-phase extraction; HVs: heavies; LTs: lights; P: pressure; RR: reflux ratio.

FIG. 2 shows a schematic of the methodology for the characterization of 1,3-bioBG and petro-1,3-BG odor active regions.

FIG. 3 shows a representative GC/MS chromatograph of 1,3-bioBG-C. X-axis: Acquisition time (minutes); Y-axis: Counts. Peaks are summarized in Table 12.

FIG. 4 shows GC/MS chromatographs with GC-O analysis of petro-1,3-BG-A, 1,3-bioBG-A, petro-1,3-BG-B, and 1,3-bioBG-B. X-axis: Acquisition time (minutes); Y-axis: Counts. Peaks are summarized in Table 13.

FIG. 5 shows GC/MS chromatographs with GC-O analysis of petro-1,3-BG-C, 1,3-bioBG-D, 1,3-bioBG-E, and 1,3-bioBG-C. X-axis: Acquisition time (minutes); Y-axis: Counts. Peaks are summarized in Table 13.

FIG. 6 shows GC/MS chromatographs with GC-O analysis of 1,3-bioBG-F, 1,3,-bioBG-G, 1,3,-bioBG-H, and 1,3,-bioBG-I. X-axis: Acquisition time (minutes); Y-axis: Counts. Peaks are summarized in Table 13.

### DETAILED DESCRIPTION OF THE INVENTION

The compositions and methods provided herein relate, in part, to the discovery of a bioderived 1,3-BG (“1,3-bioBG”) that has specific odor veins that can be used to characterize and produce a neutral odor 1,3-BG (“neutral odor 1,3-bioBG”). Odor veins are odor-active regions identified by gas chromatography-mass spectrometry (GC-MS) as having a chromatographic acquisition time that correlates with a particular odor region. The odor veins of a 1,3-bioBG on a chromatograph can be characterized and used to identify a neutral odor 1,3-bio-BG provided herein. Alternatively the chromatograph of a 1,3-bioBG can be used to remove a specific chemical from the formulation to produce a low odor 1,3-bioBG or to add a chemical to enhance an odor or fragrance in a formulation (“odorous 1,3-bioBG”). Thus, the compositions provided herein can be used in various cosmetic applications and the methods provided herein identify the optimal formulations for cosmetic use. The instant disclosure is further based, in part, on the realization that petro-BG and neutral odor 1,3-bioBG have different odor characteristics and that the different odors of petro-BG and neutral odor 1,3-bioBG are due to different impurities commonly present in petro-BG and bio-BG preparations.

Provided herein are purified neutral odor bioderived 1,3-butylene glycol products as well as processes and systems for producing such purified neutral odor 1,3-bio-BG products. Briefly, further described herein are (1) neutral odor 1,3-bioBG compositions and odor-active region characteristics; (2) methods of generating neutral odor bioderived 1,3-BG; (3) methods of purification and distillation of neutral odor 1,3-bioBG; (4) formulations; and (5) applications.

### Definitions

All definitions, as defined and used herein, should be understood to control over dictionary definitions, definitions in documents incorporated by reference, and/or ordinary meanings of the defined terms.

All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to #10%, of a given value. Alternatively, particularly with respect to systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “1,3-butylene glycol” is intended to mean a four carbon diol having a having a chemical formula of C₄H₁₀O₂ and assigned CAS No. 107-88-0. It is also known by various synonyms that are used interchangeably and include, for example, 1,3-butylene glycol BG; 1,3-BG; 1,3-butanediol; 1,3-BDO and butane-1,3-diol.

As used herein, the term “bioderived” means derived from or synthesized by a biological organism and can be considered a renewable resource since a bioderived product can be generated by a biological organism. Such a biological organism, in particular, the microbial organisms of the disclosure described here, can utilize feedstock or biomass, such as, sugars or carbohydrates obtained from an agricultural, plant, bacterial, or animal source. Alternatively, the biological organism can utilize atmospheric carbon. As used here, the term “biobased” means a product as described here that is composed of, in whole or in part, a bioderived compound of the disclosure. A biobased or bioderived product of the disclosure (e.g., bioderived 1,3-butylene glycol or bioBG) is not, and is in contrast to, a “petroleum-derived product,” where such a petroleum-derived product is derived from or synthesized from petroleum or a petrochemical feedstock. A bioderived 1,3-butylene glycol used here is a natural product as measured by the International Organization for Standardization (ISO). ISO 16128-1:2016 provides definitions for organic and natural cosmetic ingredients, and ISO 16128-2:2017 provides guidelines and criteria for determining natural, organic, or natural and organic origins of cosmetic ingredients and products.

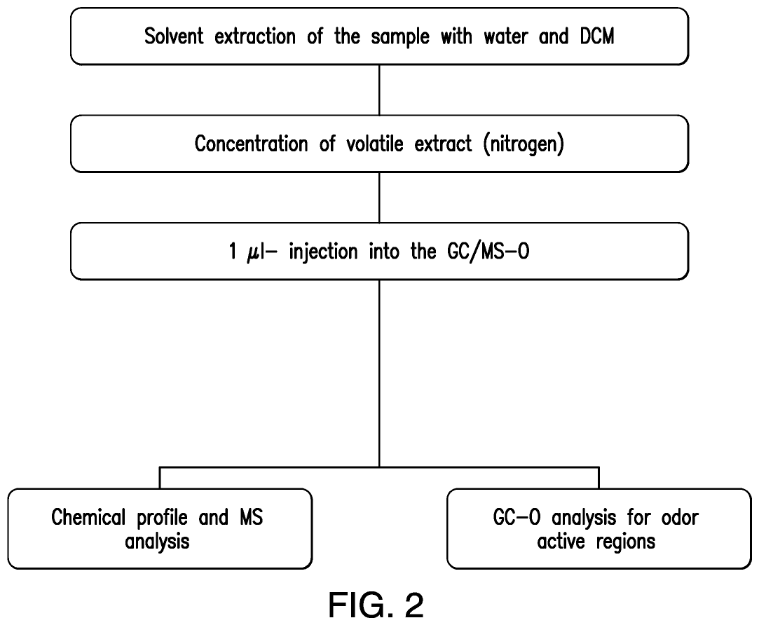

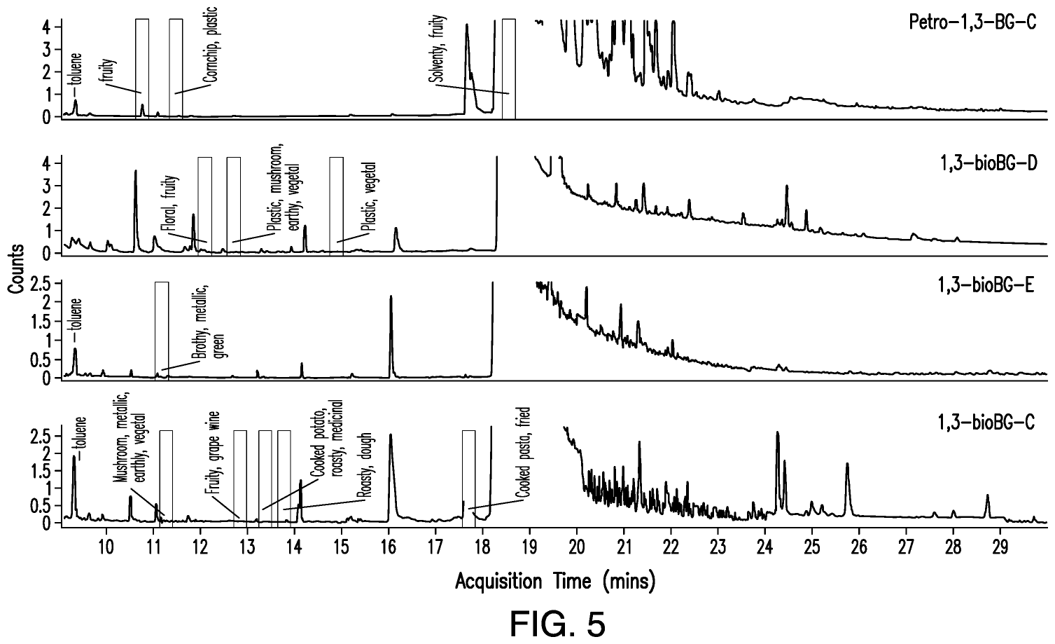

As used herein, the term “odor-active region” is intended to mean an identifiable region within a fractionated sample of 1,3-butylene glycol (1,3-BG). The fractionation method can be any method known in the art that separates components based on, for example, chemical and/or physical properties. The fractionation method exemplified in this disclosure is gas chromatography-mass spectrometry (GC-MS) as exemplified in FIGS. 2-5. The odor for the various 1,3-BG fractions is determined by a sensory odor panel composed of trained individuals and referred to herein as GC/O analysis (see, e.g., Examples 3-4).

As used herein, the term “perception threshold” is intended to mean the point at which 50% of the sensory odor panelists cannot smell the odor and corresponds to one (1) odor unit per cubic meter (1 o.u./m³).

As used herein, the terms “neutral odor” refers to a non-offensive odor to the olfactory system as determined by a subject, machine, or sensory odor panelist. A “neutral odor bioderived 1,3-BG” or a “neutral odor 1,3-bioBG” and grammatical equivalents refers to a bioderived 1,3 butylene glycol that does not have an offensive odor. For example, a sulfuric odor above the perception threshold may be considered an offensive odor as determined by sensory odor panelists.

As used herein, the term “low odor” and its grammatical equivalents refers to a non-offensive odor to the olfactory system that is below the perception threshold as determined by a subject, machine, or sensory odor panelist. The terms “low odor bioderived 1,3-butylene glycol” or “low odor bioderived 1,3-BG” or “low odor 1,3-bioBG” are used interchangeably to refer to a bioderived 1,3-butylene glycol composition that has an odor-active region that is below the perception threshold for the odor associated with the corresponding odor-active region on a GC-MS chromatograph. For example, a 1,3-bioBG composition can comprise a fruity odor-active region having a relative acquisition time of 0.594 that is below the perception threshold for that odor and would not be perceived by a panel of trainer professionals or a machine to have a fruity odor. In some cases, a low odor 1,3-bioBG is produced by removing a chemical from a neutral odor bioderived 1,3-BG composition described herein.

As used herein, the terms “odorous 1,3-bioBG” or “fragrant 1,3-bioBG” are used interchangeably to refer to a bioderived 1,3-butylene glycol composition that has an odor-active region that is above the perception threshold for the odor associated with the odor-active region. For example, a fragrant 1,3-bioBG can comprise a fruity odor-active region having a relative acquisition time of 0.594 that is above the perception threshold for that odor. In some embodiments, a chemical is added to a neutral odor 1,3-bioBG composition provided herein to produce a fragrant 1,3-bioBG.

As used herein, the term “flavoring substance” is intended to mean a material that provides or enhances a particular taste or smell. The material can include a solid, powder, liquid or gas, for example. The flavoring substance can be natural or artificial flavor. In some instances the 1,3-BG described herein can be a flavoring substance.

As used herein, the term “additive” when used in reference to food or drink is intended to mean any substance the intended use of which results in it becoming a component or otherwise affecting the characteristics of the food or drink to which it is added.

As used herein, the term “purity” refers to either chemical or chiral purity, or both.

(1) 1,3-bioBG Compositions

Bioderived 1,3-butylene glycol/is a sustainable, natural bioderived 1,3-BG (1,3-bioBG), which may be produced by non-naturally occurring microorganisms that express genes encoding enzymes that catalyze the biosynthesis of 1,3-BG. CAS No. 107-88-0 is assigned to 1,3-BG having a chemical formula of C₄H₁₀O₂. A chromatograph of a 1,3-BG composition will comprise a peak between an acquisition time of about 18 and 22 minutes. Provided herein are neutral odor bioderived 1,3-BG compositions (e.g., 1,3-bioBG-A or 1,3-bioBG-B) comprising one or more odor-active region selected from Table 1. Odor-active regions are characterized by gas-chromatography/mass spectrometry and gas chromatography-olfactometry methods. Gas chromatography-mass spectrometry (GC-MS) is an analytical method that combines the features of gas-chromatography and mass spectrometry to identify different substances within a test sample (e.g., a neutral odor 1,3-bioBG or a fragrant 1,3-bioBG). GC/MS can be used alone or in combination with gas chromatography-olfactometry (GC-O) methods and analysis. GC-O integrates the separation of volatile compounds with the detection of odor using an olfactometer. The olfactometer can be a machine, an electronic nose, a human assessor, or a database of known odor-active regions that correspond to specific GC/MS chromatograph parameters (e.g., acquisition time, counts % vs. acquisition time. intensity, or acquisition time/relative time). An odor can be determined by a panel of human or electronic assessors. One odor unit is a number where a panel of trained professionals is presented odors in decreasing dilution (increasing concentration) until detection, known as the perception threshold. The perception threshold is equal to 1 Odor Unit (o.u). One Odor Unit, (o.u./m³), is the amount of odorant(s) evaporated into one cubic meter of neutral gas. At standard conditions, it elicits a physiological response from a panel of trained professions reaching a perception threshold. The odor-active region is determined by GC-O analysis under specified conditions, wherein the relative acquisition time of 1,3-butylene glycol is 1.0.

The bioderived 1,3 composition provided herein can comprise an odor-active region on a chromatograph that is determined to be above the perception threshold for an odor by a panel of human or electronic assessors. Alternatively, a bioderived 1,3 composition provided herein can comprise an odor-active region on a chromatograph that is below the perception threshold for an odor.

Bioderived 1,3-BG compositions provided herein (e.g., a neutral odor 1,3-bioBG) can comprise any one of the odor-active regions in Table 1 and any combination thereof according to a GC-O acquisition time described herein. In some embodiments, the bioderived 1,3-BG composition comprises one or more odor-active regions from Table 1. In some embodiments, the bioderived 1,3-BG composition comprises two or more odor-active regions from Table 1. In some embodiments, the bioderived 1,3-BG composition comprises three or more odor-active regions from Table 1. In some embodiments, the bioderived 1,3-BG composition comprises four or more odor-active regions from Table 1. In some embodiments, the bioderived 1,3-BG composition comprises all five odor-active regions from Table 1. In some embodiments, the bioderived 1,3-BG composition comprises one or more odor-active regions, and further comprises a combination of 2, 3, 4, or 5, odor-active regions.

TABLE 1
Bioderived 1,3-BG composition odor-active regions.

| Relative Acquisition Time | Odor |
|---|---|
| 1.328 | roast and/or exhaust |
| 0.711 | solventy |
| 0.655 | citrus and/or peel |
| 0.628 | fruity |
| 0.594 | fruity and/or solventy |

A bioderived 1,3-BG composition provided herein can comprise one or more odors selected from Table 1 and any combination of odors. In some embodiments, the bioderived 1,3-BG composition comprises a roasty odor. In some embodiments, the bioderived 1,3-BG composition comprises an exhaust odor. In some embodiments, the bioderived 1,3-BG composition comprises a roast odor and an exhaust odor. In some embodiments, the bioderived 1,3-BG composition comprises a solventy odor. In some embodiments, the bioderived 1,3-BG composition comprises a citrus odor. In some embodiments, the bioderived 1,3-BG composition comprises a peel odor. In some embodiments, the bioderived 1,3-BG composition comprises a citrus and a peel odor. In some embodiments, the bioderived 1,3-BG composition comprises a fruity odor. In some embodiments, the bioderived 1,3-BG composition comprises a fruity and a solventy odor. The odors in Table 1 can be above the perception threshold or below the perception threshold. Exemplary combinations of odor-active regions and odors are provided below and in Table 2.

The bioderived 1,3-BG compositions provided herein (e.g., a neutral odor 1,3-bioBG) comprise an odor-active region having a relative acquisition time of 1.328 wherein the relative acquisition time of 1,3-butylene glycol is 1.0. In some embodiments, a neutral odor bioderived 1,3-BG provided herein comprises the odor-active region having a relative acquisition time of 1.328 and comprises an odor selected from roast and/or exhaust.

The bioderived 1,3-BG composition having a relative acquisition time of 1.328 can comprise an odor concentration of about 10000 odor units (o.u./m³) or less, about 1000 o.u./m³ or less, about 100 o.u./m³ or less, about 10 o.u./m³ or less, about 5 o.u./m³ or less, about 4 o.u./m³ or less, about 3 o.u./m³ or less, about 2 o.u./m³ or less, or 1 o.u./m³.

The neutral odor bioderived 1,3-BG composition can comprise an odor-active region having a relative acquisition time of 1.328 and at least one additional odor-active region, wherein the relative acquisition time of 1,3-BG is 1.0 according to GC-O analysis under specified conditions. In some embodiments, the additional odor-active region is an odor-active region listed in Table 1. The neutral odor bioderived 1,3-BG composition can include two or more odor-active regions selected from Table 1, wherein the relative acquisition time of 1,3-BG is 1.0 according to GC-O analysis under specified conditions. In some embodiments, the neutral odor bioderived 1,3 BG composition comprising an odor-active region at 1.328 relative acquisition time further comprises any one of the odor-active regions selected from: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, or (d) a solventy odor-active region having a relative acquisition time of 0.711, wherein in GC-O analysis under specified conditions the relative acquisition time of 1,3-BG is 1.0. In some embodiments, the bioderived 1,3-BG composition comprises an odor-active region at a relative acquisition time of 1.328 and a fruity and/or solventy odor-active region having a relative acquisition time of 0.594. In some embodiments, the bioderived 1,3-BG composition comprises an odor-active region at a relative acquisition time of 1.328 and a fruity odor-active region having a relative acquisition time of 0.628. In some embodiments, the bioderived 1,3-BG composition comprises an odor-active region at a relative acquisition time of 1.328 and a citrus and/or peel odor-active region having a relative acquisition time of 0.655. In some embodiments, the bioderived 1,3-BG composition comprises an odor-active region at a relative acquisition time of 1.328 and a solventy odor-active region having a relative acquisition time of 0.711. The neutral odor bioderived 1,3-BG composition can comprise an odor-active region having a relative acquisition time of 1.328; (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, and (d) a solventy odor-active region having a relative acquisition time of 0.711, wherein in GC-O analysis under specified conditions the relative acquisition time of 1,3-BG is 1.0. The above embodiments are exemplary and, given the teachings and guidance provided herein, it is understood that the neutral odor bioderived 1,3-BG described herein can include any combination of two or more odor-active regions from Table 1.

The neutral odor bioderived 1,3-BG composition can include three or more odor-active regions selected from Table 1, wherein the relative acquisition time of 1,3-BG is 1.0 according to GC-O analysis under specified conditions. In some embodiments, the bioderived 1,3-BG composition comprises an odor-active region at a relative acquisition time of 1.328; a fruity and/or solventy odor-active region having a relative acquisition time of 0.594; and a fruity odor-active region having a relative acquisition time of 0.628. In some embodiments, the bioderived 1,3-BG composition comprises odor-active regions at a relative acquisition time of 1.328; a fruity and/or solventy odor-active region having a relative acquisition time of 0.594; and a citrus and/or peel odor-active region having a relative acquisition time of 0.655. In some embodiments, the bioderived 1,3-BG composition comprises odor-active regions at a relative acquisition time of 1.328; a fruity and/or solventy odor-active region having a relative acquisition time of 0.594; and a solventy odor-active region having a relative acquisition time of 0.711. In some embodiments, the bioderived 1,3-BG composition comprises odor-active regions at a relative acquisition time of 1.328; a fruity odor-active region having a relative acquisition time of 0.628; and a citrus and/or peel odor-active region having a relative acquisition time of 0.655. In some embodiments, the bioderived 1,3-BG composition comprises odor-active regions at a relative acquisition time of 1.328; a fruity odor-active region having a relative acquisition time of 0.628; and a solventy odor-active region having a relative acquisition time of 0.711. In some embodiments, the bioderived 1,3-BG composition comprises odor-active regions at a relative acquisition time of 1.328; a citrus and/or peel odor-active region having a relative acquisition time of 0.655; and a solventy odor-active region having a relative acquisition time of 0.711. The above embodiments are exemplary and, given the teachings and guidance provided herein, it is understood that the neutral odor bioderived 1,3-BG described herein can include any combination of three or more odor-active regions from Table 1.

The neutral odor bioderived 1,3-BG composition can include four or more odor-active regions selected from Table 1, wherein the relative acquisition time of 1,3-BG is 1.0 according to GC-O analysis under specified conditions. In some embodiments, the bioderived 1,3-BG composition comprises an odor-active region at a relative acquisition time of 1.328; a fruity and/or solventy odor-active region having a relative acquisition time of 0.594; a fruity odor-active region having a relative acquisition time of 0.628; and a citrus and/or peel odor-active region having a relative acquisition time of 0.655. In some embodiments, the bioderived 1,3-BG composition comprises an odor-active region at a relative acquisition time of 1.328; a fruity and/or solventy odor-active region having a relative acquisition time of 0.594; a fruity odor-active region having a relative acquisition time of 0.628; and a solventy odor-active region having a relative acquisition time of 0.711. In some embodiments, the bioderived 1,3-BG composition comprises an odor-active region at a relative acquisition time of 1.328; a fruity odor-active region having a relative acquisition time of 0.628; a citrus and/or peel odor-active region having a relative acquisition time of 0.655; and a solventy odor-active region having a relative acquisition time of 0.711. The above embodiments are exemplary and, given the teachings and guidance provided herein, it is understood that the neutral odor bioderived 1,3-BG described herein can include any combination of four or more odor-active regions from Table 1.

The neutral odor bioderived 1,3-BG composition can include all five odor-active regions selected from Table 1, wherein the relative acquisition time of 1,3-BG is 1.0 according to GC-O analysis under specified conditions. In some embodiments, the bioderived 1,3-BG composition comprises an odor-active region at a relative acquisition time of 1.328; a fruity and/or solventy odor-active region having a relative acquisition time of 0.594; a fruity odor-active region having a relative acquisition time of 0.628; a citrus and/or peel odor-active region having a relative acquisition time of 0.655; and a solventy odor-active region having a relative acquisition time of 0.711.

Neutral odor bioderived 1,3-BG compositions provided herein can comprise an odor concentration that is above the perception threshold as determined by GC-O analysis under specified conditions. As described above the odor concentration is the amount of odorant(s) evaporated into one cubic meter of neutral gas (odor units per cubic meter or o.u./m³). The neutral odor bioderived 1,3-BG compositions comprising one or more odor-active regions having a relative acquisition time listed in Table 1 can comprise an odor concentration of at least about 1 o.u/m³. In some embodiments, a bioderived 1,3-BG composition having one or more odor-active regions having a relative acquisition time listed in Table 1 comprises an odor concentration between at least about 1 o.u/m³ up to 10000 o.u/m³.

In some embodiments, the bioderived 1,3-BG composition comprises an odor-active region at a relative acquisition time of 1.328 having a roasty and/or exhaust odor, wherein the odor concentration is at least about 1 o.u/m³.

Further provided herein are low odor bioderived 1,3-BG compositions that comprise one or more odor-active region having a relative acquisition time provided in Table 1 and comprise an odor concentration below the perception threshold or below 1 o.u/m³. In some embodiments, the bioderived 1,3-BG provided herein can comprise an odor-active region having a relative acquisition time of 1.328 that is below the perception threshold. In some embodiments, the low odor bioderived 1,3-BG composition comprises (a) a fruity and/or solventy odor-active region having a relative acquisition time of 0.594 that is below the perception threshold; (b) the fruity odor-active region having a relative acquisition time of 0.628 that is below the perception threshold; (c) the citrus and/or peel odor-active region having a relative acquisition time of 0.655 that is below the perception threshold, or (d) the solventy odor-active region having a relative acquisition time of 0.711 is below the perception threshold or any combination thereof. In some embodiments, the low odor bioderived 1,3-BG composition comprises an odor-active region having a relative acquisition time of 1.328; (a) a fruity and/or solventy odor-active region having a relative acquisition time of 0.594 that is below the perception threshold; (b) the fruity odor-active region having a relative acquisition time of 0.628 that is below the perception threshold; (c) the citrus and/or peel odor-active region having a relative acquisition time of 0.655 that is below the perception threshold, or (d) the solventy odor-active region having a relative acquisition time of 0.711 is below the perception threshold or any combination thereof.

Neutral odor 1,3-bioBG compositions provided herein can comprise any one or more odor-active regions selected from Table 1 and comprise any odor listed in Table 1. The neutral odor 1,3-bioBG composition can comprise one or more, two or more, three or more, four or more, or all five odor active-regions and odors from Table 1. The odor or odors detected in the 1,3 bioBG composition can be above the perception threshold or below the perception threshold. Further provided herein are neutral odor bioderived 1,3-butylene glycol (1,3-bioBG) compositions, wherein the composition comprises any one or more of: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, or (d) a solventy odor-active region having a relative acquisition time of 0.711, wherein in GC-O analysis under specified conditions the relative acquisition time of 1,3-BG is 1.0. In some embodiments, a neutral odor 1,3-bioBG composition comprises (a) a fruity and/or solventy odor-active region having a relative acquisition time of 0.594. In some embodiments, a neutral odor 1,3-bioBG composition comprises (b) a fruity odor-active region having a relative acquisition time of 0.628. In some embodiments, a neutral odor 1,3-bioBG composition comprises (c) a citrus and/or peel odor-active region having a relative acquisition time of 0.655. In some embodiments, a neutral odor 1,3-bioBG composition comprises (d) a solventy odor-active region having a relative acquisition time of 0.711.

In some embodiments, the neutral odor 1,3-bioBG composition comprises two or more odor-active regions and any two odors from Table 1. In some embodiments, the bioderived 1,3-BG composition comprises: (a) a fruity and/or solventy odor-active region having a relative acquisition time of 0.594; and (b) a fruity odor-active region having a relative acquisition time of 0.628. In some embodiments, the bioderived 1,3-BG composition comprises: (a) a fruity and/or solventy odor-active region having a relative acquisition time of 0.594; and (c) a citrus and/or peel odor-active region having a relative acquisition time of 0.655. In some embodiments, the bioderived 1,3-BG composition comprises: (a) a fruity and/or solventy odor-active region having a relative acquisition time of 0.594; and (d) a solventy odor-active region having a relative acquisition time of 0.711. In some embodiments, the bioderived 1,3-BG composition comprises: (b) a fruity odor-active region having a relative acquisition time of 0.628; and (c) a citrus and/or peel odor-active region having a relative acquisition time of 0.655. In some embodiments, the bioderived 1,3-BG composition comprises: (b) a fruity odor-active region having a relative acquisition time of 0.628; and (d) a solventy odor-active region having a relative acquisition time of 0.711. In some embodiments, the bioderived 1,3-BG composition comprises: (c) a citrus and/or peel odor-active region having a relative acquisition time of 0.655; (d) a solventy odor-active region having a relative acquisition time of 0.711. The above embodiments are exemplary and, given the teachings and guidance provided herein, it is understood that the neutral odor bioderived 1,3-BG described herein can include any combination of two or more odor-active regions from Table 1.

In some embodiments, the bioderived 1,3-BG composition comprises: (a) a fruity and/or solventy odor-active region having a relative acquisition time of 0.594; and an odor-active region having a relative acquisition time of 1.328. In some embodiments, the bioderived 1,3-BG composition comprises: (a) a fruity and/or solventy odor-active region having a relative acquisition time of 0.594; and a roasty and/or exhaust odor-active region having a relative acquisition time of 1.328.

In some embodiments, the bioderived 1,3-BG composition comprises: (b) a fruity odor-active region having a relative acquisition time of 0.628; and an odor-active region having a relative acquisition time of 1.328. In some embodiments, the bioderived 1,3-BG composition comprises: (b) a fruity odor-active region having a relative acquisition time of 0.628; and a roasty and/or exhaust odor-active region having a relative acquisition time of 1.328.

In some embodiments, the bioderived 1,3-BG composition comprises: (c) a citrus and/or peel odor-active region having a relative acquisition time of 0.655; and an odor-active region having a relative acquisition time of 1.328. In some embodiments, the bioderived 1,3-BG composition comprises: (c) a citrus and/or peel odor-active region having a relative acquisition time of 0.655; and a roasty and/or exhaust odor-active region having a relative acquisition time of 1.328.

In some embodiments, the bioderived 1,3-BG composition comprises: (d) a solventy odor-active region having a relative acquisition time of 0.711; and an odor-active region having a relative acquisition time of 1.328. In some embodiments, the bioderived 1,3-BG composition comprises: (d) a solventy odor-active region having a relative acquisition time of 0.711; and a roasty and/or exhaust odor-active region having a relative acquisition time of 1.328.

In some embodiments, the neutral odor 1,3-bioBG composition comprises three or more odor-active regions and any three odors from Table 1. In some embodiments, a 1,3-bioBG composition provided herein comprises: (a) a fruity and/or solventy odor-active region having a relative acquisition time of 0.594; (b) a fruity odor-active region having a relative acquisition time of 0.628; and (c) a citrus and/or peel odor-active region having a relative acquisition time of 0.655. In some embodiments, a 1,3-bioBG composition comprises: (a) a fruity and/or solventy odor-active region having a relative acquisition time of 0.594; (b) a fruity odor-active region having a relative acquisition time of 0.628; and (d) a solventy odor-active region having a relative acquisition time of 0.711. In some embodiments, a 1,3-bioBG composition provided herein comprises (b) a fruity odor-active region having a relative acquisition time of 0.628; (c) a citrus and/or peel odor-active region having a relative acquisition time of 0.655; and (d) a solventy odor-active region having a relative acquisition time of 0.711. In some embodiments, a 1,3-bioBG composition provided herein comprises: (a) a fruity and/or solventy odor-active region having a relative acquisition time of 0.594; (b) a fruity odor-active region having a relative acquisition time of 0.628; and an odor-active region having a relative acquisition time of 1.328. In some embodiments, a 1,3-bioBG composition provided herein comprises: (a) a fruity and/or solventy odor-active region having a relative acquisition time of 0.594; (b) a fruity odor-active region having a relative acquisition time of 0.628; and a roasty and/or exhaust odor-active region having a relative acquisition time of 1.328. In some embodiments, a 1,3-bioBG composition provided herein comprises: (a) a fruity and/or solventy odor-active region having a relative acquisition time of 0.594; (c) a citrus and/or peel odor-active region having a relative acquisition time of 0.655; and an odor-active region having a relative acquisition time of 1.328. In some embodiments, a 1,3-bioBG composition provided herein comprises: (a) a fruity and/or solventy odor-active region having a relative acquisition time of 0.594; (c) a citrus and/or peel odor-active region having a relative acquisition time of 0.655; and a roasty and/or exhaust odor-active region having a relative acquisition time of 1.328. In some embodiments, a 1,3-bioBG composition provided herein comprises: (a) a fruity and/or solventy odor-active region having a relative acquisition time of 0.594; (d) a solventy odor-active region having a relative acquisition time of 0.711; and an odor-active region having a relative acquisition time of 1.328. In some embodiments, a 1,3-bioBG composition provided herein comprises: (a) a fruity and/or solventy odor-active region having a relative acquisition time of 0.594; (d) a solventy odor-active region having a relative acquisition time of 0.711; and a roasty and/or exhaust odor-active region having a relative acquisition time of 1.328. In some embodiments, a 1,3-bioBG composition provided herein comprises: (b) a fruity odor-active region having a relative acquisition time of 0.628; (c) a citrus and/or peel odor-active region having a relative acquisition time of 0.655; and an odor-active region having a relative acquisition time of 1.328. In some embodiments, a 1,3-bioBG composition provided herein comprises: (b) a fruity odor-active region having a relative acquisition time of 0.628; (c) a citrus and/or peel odor-active region having a relative acquisition time of 0.655; and a roasty and/or exhaust odor-active region having a relative acquisition time of 1.328. In some embodiments, a 1,3-bioBG composition provided herein comprises: (b) a fruity odor-active region having a relative acquisition time of 0.628; (d) a solventy odor-active region having a relative acquisition time of 0.711; and an odor-active region having a relative acquisition time of 1.328. In some embodiments, a 1,3-bioBG composition provided herein comprises: (b) a fruity odor-active region having a relative acquisition time of 0.628; (d) a solventy odor-active region having a relative acquisition time of 0.711; and a roasty and/or exhaust odor-active region having a relative acquisition time of 1.328. In some embodiments, a 1,3-bioBG composition provided herein comprises: (c) a citrus and/or peel odor-active region having a relative acquisition time of 0.655; (d) a solventy odor-active region having a relative acquisition time of 0.711; and an odor-active region having a relative acquisition time of 1.328. In some embodiments, a 1,3-bioBG composition provided herein comprises (c) a citrus and/or peel odor-active region having a relative acquisition time of 0.655; (d) a solventy odor-active region having a relative acquisition time of 0.711; and a roasty and/or exhaust odor-active region having a relative acquisition time of 1.328. The above embodiments are exemplary and, given the teachings and guidance provided herein, it is understood that the neutral odor bioderived 1,3-BG described herein can include any combination of three or more odor-active regions from Table 1.

In some embodiments, the neutral odor 1,3-bioBG composition comprises four or more odor-active regions and any four odors from Table 1. In some embodiments, a 1,3-bioBG composition comprises (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655; and (d) a solventy odor-active region having a relative acquisition time of 0.711. In some embodiments, a 1,3-bioBG composition comprises (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655; and an odor-active region having a relative acquisition time of 1.328. In some embodiments, a 1,3-bioBG composition comprises (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655; and a roasty and/or exhaust odor-active region having a relative acquisition time of 1.328. In some embodiments, a 1,3-bioBG composition comprises (a) a fruity and/or solventy odor-active region having a relative acquisition time of 0.594; (b) a fruity odor-active region having a relative acquisition time of 0.628; (d) a solventy odor-active region having a relative acquisition time of 0.711; and an odor-active region having a relative acquisition time of 1.328. In some embodiments, a 1,3-bioBG composition comprises (a) a fruity and/or solventy odor-active region having a relative acquisition time of 0.594; (b) a fruity odor-active region having a relative acquisition time of 0.628; (d) a solventy odor-active region having a relative acquisition time of 0.711; and a roasty and/or exhaust odor-active region having a relative acquisition time of 1.328. In some embodiments, a 1,3-bioBG composition comprises: (b) a fruity odor-active region having a relative acquisition time of 0.628; (c) a citrus and/or peel odor-active region having a relative acquisition time of 0.655; (d) a solventy odor-active region having a relative acquisition time of 0.711; and an odor-active region having a relative acquisition time of 1.328. In some embodiments, a 1,3-bioBG composition comprises: (b) a fruity odor-active region having a relative acquisition time of 0.628; (c) a citrus and/or peel odor-active region having a relative acquisition time of 0.655; (d) a solventy odor-active region having a relative acquisition time of 0.711; and a roasty and/or exhaust odor-active region having a relative acquisition time of 1.328. The above embodiments are exemplary and, given the teachings and guidance provided herein, it is understood that the neutral odor bioderived 1,3-BG described herein can include any combination of four or more odor-active regions from Table 1.

A 1,3-bioBG composition provided herein can comprise all five odor-active regions from Table 1 and any one or more of the odors listed in Table 1. The odor(s) can be above the perception threshold or below the perception threshold. In some embodiments, the bioderived 1,3-butylene glycol (1,3-bioBG) composition comprises: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, and/or (d) a solventy odor-active region having a relative acquisition time of 0.711, and further comprises an odor-active region having a relative acquisition time of 1.328, wherein in GC-O analysis under specified conditions the relative acquisition time of 1,3-BG is 1.0. In some embodiments, the bioderived 1,3-butylene glycol (1,3-bioBG) composition comprises: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, or (d) a solventy odor-active region having a relative acquisition time of 0.711; (e) a roasty and/or exhaust odor-active region having a relative acquisition time of 1.328.

Additional exemplary combinations of odor-active region acquisition times and odors that can be present in the bioderived 1,3-BG compositions described herein are provided in Table 2 below. The “x” indicates the presence of the odor-active region and/or odor in a neutral odor 1,3-bioBG composition.

TABLE 2
Exemplary combinations of odors and odor-active regions.

| Acquisition Time | 0.594 | 0.628 | 0.655 | 0.711 | 1.328 |
|---|---|---|---|---|---|
| 0.594 | | X | X | X | X |
| 0.628 | X | | X | X | X |
| 0.655 | X | X | | X | X |
| 0.711 | X | X | X | | X |
| 1.328 | X | X | X | X | |
| | fruity and/ | | citrus and/ | | roast and/ |
| Odors | or solventy | fruity | or peel | solventy | or exhaust |
| fruity and/or | | X | X | X | X |
| solventy | | | | | |
| fruity | X | | X | X | X |
| citrus and/or peel | X | X | | X | X |
| solventy | X | X | X | | X |
| roast and/or | X | X | X | X | |
| exhaust | | | | | |
| Acquisition | fruity and/ | | citrus and/ | | roast and/ |
| Time/Odor | or solventy | fruity | or peel | solventy | or exhaust |
| 0.594 | | X | X | X | X |
| 0.628 | X | | X | X | X |
| 0.655 | X | X | | X | X |
| 0.711 | X | X | X | | X |
| 1.328 | X | X | X | X | |

The neutral odor bioderived 1,3-BG compositions can comprise different concentrations of odors per cubic meter for each odor associated with an acquisition time as determined by GC-O.

In some embodiments, a bioderived 1,3-BG composition provided herein comprises (a) the fruity and/or solventy odor-active region having a relative acquisition time of 0.594 has an odor concentration between at least about 1 o.u./m³ up to 10000 o.u./m³; (b) the fruity odor-active region having a relative acquisition time of 0.628 has an odor concentration between at least about 1 o.u./m³ up to 10000 o.u./m³; (c) the citrus and/or peel odor-active region having a relative acquisition time of 0.655 has an odor concentration between at least about 1 o.u./m³ up to 10000 o.u./m³; or (d) the solventy odor-active region having a relative acquisition time of 0.711 has an odor concentration between at least about 1 o.u./m³ up to 10000 o.u./m³.

Further provided herein are 1,3-bioBG compositions (e.g., a neutral odor 1,3-bioBG or a low odor 1,3-bioBG) comprising an odor concentration at any one or more odor-active regions set out in Table 1; and one or more additional odor-active regions, wherein the one or more additional odor-active regions comprise an odor concentration below the perception threshold. The odor-active region can be detected by GC-MS and/or GC-O but is not detected by a trained a panel of human or electronic assessors.

A bioderived 1,3 BG composition provided herein can comprise an odor concentration at or lower than the perception threshold at any one or more odor-active regions having a relative acquisition time selected from the group consisting of: 0.544, 0.583, 0.589, 0.594, 0.60, 0.605, 0.616, 0.622, 0.628, 0.633, 0.655, 0.678, 0.683, 0.689, 0.694, 0.70, 0.705, 0.711, 0.739, 0.744, 0.750, 0.755, 0.761, 0.767, 0.80, 0.816, 0.817, 0.833, 0.872, 0.928, 0.972, 0.983, 1.017, 1.033, 1.117, 1.139, 1.211 and 1.405, wherein in GC-O analysis under specified conditions the relative acquisition time of 1,3-BG is 1.0. The bioderived 1,3-BG composition (e.g., a neutral odor 1,3-bioBG or a low odor 1,3-bioBG) can comprise an odor concentration at or lower than the perception threshold for an odor selected from the group consisting of: fruity, sweet, green, pungent, cheesy, solventy, mushroom, metallic, brothy, waxy, earthy, vegetal, corn chip, plastic, grassy, floral, citrus, peel, sulfuric, floral (rosy), acrylic, pungent, grape wine, medicinal, roasty, nutty, bready, cooked potato, pyrazine-like, plastic-like, dough, cooked rice, stinky, green floral, burnt plastic, cooked pasta, fried, acid, potato, pickled plum, green bell pepper, celery, exhaust, lactone-like and estery as set forth in Table 13. In some embodiments, the bioderived 1,3-BG composition (e.g., a neutral odor 1,3-bioBG or a low odor 1,3-bioBG) does not comprise sweet, green, pungent, cheesy, mushroom, metallic, brothy, waxy, earthy, vegetal, corn chip, plastic, grassy, floral, sulfuric, floral (rosy), acrylic, pungent, grape wine, medicinal, nutty, bready, cooked potato, pyrazine-like, plastic-like, dough, cooked rice, stinky, green floral, burnt plastic, cooked pasta, fried, acid, potato, pickled plum, green bell pepper, celery, exhaust, lactone-like or estery odors when GC-O analysis is performed under specified conditions. In some embodiments, the bioderived 1,3-BG composition (e.g., a neutral odor 1,3-bioBG or a low odor 1,3-bioBG) comprises any one or more odor-active regions set out in Table 1; and does not comprise sweet, green, pungent, cheesy, mushroom, metallic, brothy, waxy, earthy, vegetal, corn chip, plastic, grassy, floral, sulfuric, floral (rosy), acrylic, pungent, grape wine, medicinal, nutty, bready, cooked potato, pyrazine-like, plastic-like, dough, cooked rice, stinky, green floral, burnt plastic, cooked pasta, fried, acid, potato, pickled plum, green bell pepper, celery, lactone-like or estery odors when GC-O analysis is performed under specified conditions.

A bioderived 1,3-BG composition can comprise any one or more odor-active regions provided herein in any combination. The odor-active region and odor associated with the odor active region can be above the perception threshold or below the perception threshold as determined by GC-O analysis under specified conditions. In some embodiments, a bioderived 1,3-BG composition comprises a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30 31, 32, 33, 34, 35, 36 or 37 odor-active regions having a relative acquisition time selected from: 0.544, 0.583, 0.589, 0.594, 0.60, 0.605, 0.616, 0.622, 0.628, 0.633, 0.655, 0.678, 0.683, 0.689, 0.694, 0.70, 0.705, 0.711, 0.739, 0.744, 0.750, 0.755, 0.761, 0.767, 0.80, 0.816, 0.817, 0.833, 0.872, 0.928, 0.972, 0.983, 1.017, 1.033, 1.117, 1.139, 1.211 and 1.405. In some embodiments, a bioderived 1,3-BG composition comprises an odor concentration at or lower than the perception threshold at one or more odor-active regions having a relative acquisition time selected from the group consisting of 0.544, 0.583, 0.589, 0.594, 0.60, 0.605, 0.616, 0.622, 0.628, 0.633, 0.655, 0.678, 0.683, 0.689, 0.694, 0.70, 0.705, 0.711, 0.739, 0.744, 0.750, 0.755, 0.761, 0.767, 0.80, 0.816, 0.817, 0.833, 0.872, 0.928, 0.972, 0.983, 1.017, 1.033, 1.117, 1.139, 1.211 and 1.405; and can further comprise any one or more of: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, (d) a solventy odor-active region having a relative acquisition time of 0.711; or (e) an odor-active region having a relative acquisition time of 1.328, wherein any one or more of (a)-(e) is at or lower than the perception threshold. In some embodiments, a bioderived 1,3-BG composition comprises an odor concentration at or lower than the perception threshold at one or more odor-active regions having a relative acquisition time selected from the group consisting of 0.544, 0.583, 0.589, 0.594, 0.60, 0.605, 0.616, 0.622, 0.628, 0.633, 0.655, 0.678, 0.683, 0.689, 0.694, 0.70, 0.705, 0.711, 0.739, 0.744, 0.750, 0.755, 0.761, 0.767, 0.80, 0.816, 0.817, 0.833, 0.872, 0.928, 0.972, 0.983, 1.017, 1.033, 1.117, 1.139, 1.211 and 1.405; and further comprises any one or more of: (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655, (d) a solventy odor-active region having a relative acquisition time of 0.711; or (e) a roasty and/or exhaust odor-active region having a relative acquisition time of 1.328, wherein any one or more of (a)-(e) is at or lower than the perception threshold.

In some embodiments, a bioderived 1,3-BG composition comprises an odor concentration at the one or more odor-active regions is at least 50% lower than the perception threshold. In some embodiments, a bioderived 1,3-BG composition comprises the odor concentration at the one or more odor-active regions is between 55% and 100% lower than the perception threshold. A bioderived 1,3-BG composition can comprise an odor concentration at the one or more odor-active regions that are at least 55% lower, at least 60% lower, at least 65% lower, at least 70% lower, at least 75% lower, at least 80% lower, at least 85% lower, at least 90% lower, at least 95% lower, up to 100% lower than the perception threshold.

### (2) 1,3-BG Formulations

Provided herein are compositions, wherein the compositions comprise a bioderived 1,3-butylene glycol (e.g., neutral odor 1,3-bioBG); and a product ingredient. The product ingredient can be a cosmetic, fragrance, personal care product ingredient, flavoring substance, food additive, or drink additive. In some embodiments, a neutral odor bioderived 1,3-BG composition provided herein is included in a composition as a humectant, solvent, viscosity controlling agent, diluent, fragrance enhancer or flavor enhancer.

Neutral odor bioderived 1,3-BG compositions provided herein can be used in cosmetics as an ingredient that acts as an emollient, a humectant, an agent that prevents crystallization of insoluble ingredients, a solubilizer for less-water-soluble ingredients such as fragrances, and as an anti-microbial agent and preservative. In some embodiments, a composition comprising a neutral odor 1,3-bioBG provided is formulated as a cosmetic. Non-limiting examples of cosmetics include: hair and bath products, facial makeup, eye makeup, eye liner, eye shadow, eye serum, eye primer, mascara, hyaluronic acid moisturizer, firming serum, glycolic acid toning solution, retinol cream, brow liner, concealer, foundation, beauty balm cream, color correcting cream, face tint, facial moisturizer, facial mist, sunscreen, bronzer, facial primer, anti-ageing cream, ingrown hair treatments, redness relief serum or cream, makeup remover, facial or body toner, facial cleanser, acne treatment, facial peel, facial mask, night cream, face oil, tinted moisturizer, setting spray, contour, highlighter, lipstick, lip gloss, lip balm, lip stain, lip plumper, and lip masks. Additional examples of cosmetics include, for example, those described in the Cosmetic Ingredient Review board's report: “Final Report on the Safety Assessment of Butylene Glycol, Hexylene Glycol, Ethoxydiglycol, and Dipropylene Glycol”, Journal of the American College of Toxicology, Volume 4, Number 5, 1985, which is incorporated herein by reference in its entirety). This report provides specific uses and concentrations of 1,3-BG in cosmetics. See, e.g., Report, Table 2 (“Product Formulation Data”). While the Report describes uses of petro-1,3-BG, the neutral odor bioderived 1,3-BG compositions provided herein are expected to be superior products to petro-BG compositions, at least because of their improved purity profile and preferable odor characteristics.

In some embodiments, a composition comprising a 1,3-bioBG (e.g., a neutral odor 1,3-bioBG or a low odor 1,3-bioBG) is formulated as a personal care product. Exemplary personal care products include, but are not limited to, hair care productions, aromatherapy compositions for baths, chest rubs, compresses, facial steams, massage oils, respiratory inhalations, room sprays, candles, clothing/linen sprays; stationery sprays; personal care compositions such as body/facial sprays, facial serums, creams, lotions, deodorant/anti-perspirants, shampoos, conditioners, body washes; and wipes. Compositions can be formulated, for example, for topical administration (e.g., as an extract or parfum, eau de parfum, eau de toilette, eau de cologne, eau de fraiche, aftershave, cream, gel, lotion, ointment, oil (e.g., essential oil, massage oil, aromatherapy), shampoo, hair conditioner, hairspray, shower gel, soap, deodorant, anti-perspirant) or in any other formulation that utilizes a fragrance (e.g., air freshener, room and/or carpet deodorizer/freshener, laundry detergent, fabric softener, dryer sheet). The bioBG of the disclosure is a multifunctional ingredient, which may be used in a variety of personal care products and cosmetics, including serums, body scrubs, masks, cleansers, and the like, in combination with at least one fragrance ingredient, or one or more fragrance ingredients, to result in a desired scent or combination of scents.

In some embodiments, the bioderived 1,3-BG personal care compositions are hair care compositions. Hair care compositions include but not limited to shampoos, conditioners, hair treatment creams, hair masks, aerosols, hair gels, hair sprays, hair styling creams and serums, pomade, texturizing spray, hair heat protectants, scalp treatments, anti-frizz treatments, hair primer, dry and wet spray shampoos, air dry hair creams, hair oil, volumizer, set lotions, split end repair cream, defining cream, hair dyes, root cover up powder, blow styling lotions and sprays, hair relaxing compositions, hair smoothing lotions, and mousses. The hair care compositions comprise an effective amount of 1,3-bioBG in a cosmetically acceptable medium. An effective amount of 1,3-bioBG for use in a hair care composition is herein defined as a proportion of from about 1% to about 30% by weight relative to the total weight of the composition. Components of a cosmetically acceptable medium for hair care compositions are described by Omura et al. in U.S. Pat. No. 6,139,851 and Cannell et al. in U.S. Pat. No. 6,013,250, both of which are incorporated herein by reference. For example, these hair care compositions can be aqueous, alcoholic or aqueous-alcoholic solutions, the alcohol preferably being a monohydric alcohol such as ethanol or isopropanol, in a proportion of from about 1 to about 75% by weight relative to the total weight for the aqueous-alcoholic solutions. The hair care compositions may also contain other polyhydric alcohols including, but not limited to, ethylene glycol, propylene glycol, 1,3-butylene glycol, glycerol (glycerine), sorbitol, 2-methyl-1,3-propanediol, and polyethylene glycol. Additionally, the hair care compositions may contain one or more conventional cosmetic or dermatological additives or adjuvants.

In some embodiments, a composition comprising a bioderived 1,3-butylene glycol provided herein (e.g., a neutral odor 1,3-bioBG or a low odor 1,3-bioBG) and/or a product ingredient provided herein is a fragrance. Non-limiting examples of fragrance compositions include but are not limited to: Eau Fraiche: 1%-3%; Eau de Cologne: 3%-8%; Eau de Toilette: 5%-10%; Eau de Parfum: 10%-20%; Extrait or Parfum: 15%-30%; Solid Perfume (Parfum Solide): 20%-45%, C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; Vertofix Coeur; or combinations thereof. Other embodiments may be directed to compositions comprising 1,3-BG (such as but not limited to, 1,3-bioBG) and one or more fragrance ingredients that express similar odors or scents to those presented here, or when in combination with other fragrance ingredients express similar odors, scents, or fragrance profiles. Neutral odor bioderived 1,3-BG can reduce loss of aromas from essential oils, preserve against spoilage by microorganisms, and be used as a solvent for benzoates.

A bioderived 1,3-BG composition provided herein (e.g., a neutral odor 1,3-bioBG or a low odor 1,3-bioBG) has or can have further food related uses including use directly as a food source, a food ingredient, a flavoring agent, a solvent or solubilizer for flavoring agents, a stabilizer, an emulsifier, and an anti-microbial agent and preservative. In some embodiments, a composition comprising a neutral odor 1,3-bioBG provided herein is formulated as a flavoring substance. In some embodiments, a composition provided herein comprises a food additive or a drink additive. Flavoring agents are substances added to foods, beverages, cosmetics, pharmaceuticals, or medicines to improve the quality of the taste if such compositions. Non-limiting examples of a flavoring substance is a flavoring oil (e.g., orange oil), a food coloring agents, preservatives, or spices.

In some embodiments, a composition comprising a bioderived 1,3-BG composition provided herein (e.g., a neutral odor 1,3-bioBG or a low odor 1,3-bioBG) is formulated as a food composition. The food composition can be for consumption by a mammal, for example by a human or a non-human mammal. A neutral odor bioderived 1,3-BG provided herein can be formulated as a dietary supplement or a medical food. In some embodiments, a neutral odor bioderived 1,3-BG composition provided herein comprises a food ingredient. A food ingredient is any product, composition, or a component of a food known to have or disclosed as having a nutritional effect. Food can include various meats (e.g., beef, pork, poultry, fish, etc.), dairy products (e.g., milk, cheese, eggs), fruits, vegetables, cereals, breads, etc., and components thereof. Food can be fresh or preserved, e.g., by canning, dehydration, freezing, or smoking. Food can be provided in raw, unprepared and/or natural states or in cooked, prepared, and/or combined states. In some embodiments, the food ingredient is selected from the group consisting of: fat, carbohydrates, protein, fiber, nutritional balancing agent, and mixtures thereof.

Neutral odor bioderived 1,3-BG compositions can have various uses, including a stabilizer, an emulsifier, and an anti-microbial agent, and preservative. Neutral odor bioderived 1,3-BG can be used in pharmaceutical compositions as a parenteral drug solvent. In some embodiments, the neutral odor bioderived 1,3-BG is admixed with one or more humectants, solvents, viscosity controlling agents, diluents, fragrance enhancers or flavor enhancers. Exemplary humectants include but are not limited to: Zemea® (also known as USP-FCC 1, 3 propanediol; from DuPont Tate & Lyle BioProducts, 198 Blair Bend Drive, Louden, Tenn. 37774, (866) 404-7933), glycerol, sorbitol, polydextrose and propylene glycol. The bioderived 1,3-BG compositions provided herein may also contain moisturizers, preferably at about 0.1 to about 30%, more preferably at about 0.5 to about 25%, most preferably at about 1 to about 20% by weight of the total composition. These moisturizers include water-soluble, low molecular weight moisturizers, fat-soluble, low molecular weight moisturizers, water-soluble, high molecular weight moisturizers and fat-soluble, high molecular weight moisturizers. Suitable water-soluble, low molecular weight moisturizers include, but are not limited to, serine, glutamine, sorbitol, mannitol, pyrrolidone-sodium carboxylate, glycerin, propylene glycol, 1,3-butylene glycol, ethylene glycol, polyethylene glycol (polymerization degree n=2 or more), polypropylene glycol (polymerization degree n=2 or more), polyglycerin (polymerization degree n=2 or more), lactic acid and lactate. The water soluble, low molecular weight moisturizer can also be biologically-derived 1,3-propanediol. Suitable fat-soluble, low molecular weight moisturizers include, but are not limited to, cholesterol and cholesterol ester. Suitable water-soluble, high molecular weight moisturizers include, but are not limited to, carboxyvinyl polymers, polyaspartate, tragacanth, xanthan gum, methyl cellulose, hydroxymethyl cellulose, hydroxyethyl cellulose, hydroxypropyl cellulose, carboxymethyl cellulose, water-soluble chitin, chitosan and dextrin. Suitable fat-soluble, high molecular weight moisturizers include, but are not limited to, polyvinylpyrrolidone-eicosene copolymers, polyvinylpyrrolidone-hexadecene copolymers, nitrocellulose, dextrin fatty acid ester and high molecular silicone. Bioderived 1,3-BG is or can be used in the pharmaceutical industry as a parenteral drug solvent. Among the acceptable vehicles and solvents that may be employed are water, Ringer's solution, U.S.P., and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use. In some embodiments, the bioderived 1,3-BG compositions provided herein comprise UV absorbing organic sunscreens.

A bioderived 1,3-BG composition provided herein (e.g., a neutral odor 1,3-bioBG or a low odor 1,3-bioBG) can be formulated with a pharmaceutically acceptable carrier. Exemplary carriers and excipients can include but are not limited to: dextrose, sodium chloride, sucrose, lactose, cellulose, xylitol, sorbitol, maltitol, gelatin, polymers, polyethylene glycol (PEG), polyvinylpyrrolidone (PVP), and any combination thereof. In some embodiments, an excipient such as dextrose or sodium chloride can be at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%. A bioderived 1,3-BG composition provided herein (e.g., a neutral odor 1,3-bioBG or a low odor 1,3-bioBG) can be suitable for pharmaceutical formulations for transdermal application of an agent or compound. Formulations for topical application, e.g., to the skin and eyes, are preferably aqueous solutions, ointments, creams or gels well-known in the art. The formulations may contain solubilizers, stabilizers, tonicity enhancing agents, buffers and preservatives to assist passage of an agent or compound through the skin of a subject.

### (3) Methods of Making 1,3-BG

Provided herein is are neutral odor bioderived 1,3-butylene glycol compositions produced by a genetically engineered microbial organism or a population thereof. The methods and compositions for producing a bioderived 1,3-BG through culturing of non-naturally occurring microbial organisms are described in, for example, WO 2010/127319, WO 2011/071682, and WO 2012/177619, which disclose methods, vectors, strains, and culturing materials and conditions for producing bioderived 1,3-BG. WO 2018/183628 and WO 2018/183664, specifically disclose engineered organisms and enzymes producing bioderived 1,3-BG, as well as characterization of bioderived 1,3-BG, each of which is incorporated by reference in its entirety. The compositions, methods, and uses disclosed herein, in any or all embodiments, are directed to 1,3-BG, which may include any type or form of 1,3-BG, including but not limited to 1,3-BG; bioBG; mixtures of 1,3-BG and bioBG; (R)-1,3-BG; (S)-1,3-BG; mixtures of (R)-1,3-BG and(S)-1,3-BG; or any combinations or mixtures of the aforementioned.

Host microbial organisms for the production of a neutral odor bioderived 1,3-BG provided herein can be selected from, and the non-naturally occurring microbial organisms generated in, for example, e.g., bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichiapastoris. E. coli is a particularly useful host organism since it is a well characterized microbial organism suitable for genetic engineering. Other particularly useful host organisms include yeast such as Saccharomyces cerevisiae.

Methods for constructing and testing the expression levels of a non-naturally occurring 1,3-butylene glycol-producing host can be performed, for example, by recombinant methods and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

Additional methods of producing a 1,3-BG product are known in the art and include, e.g., U.S. Pat. No. 6,376,725, which is incorporated herein by reference in its entirety, describes methods of liquid phase hydrogenation of acetaldol (3-hydroxybutanal or aldol) with a Raney nickel catalyst resulting in 1,3-butylene glycol. Another method of producing 1,3-butylene glycol is described as having three steps: aldol condensation of acetaldehyde to aldoxane, then decomposition of the aldoxane resulting in paraldol, which is then hydrogenated to finally produce 1,3-butylene glycol (as described in, e.g., U.S. Pat. Nos. 5,345,004; 5,583,270, which are incorporated herein by reference in their entirety).

### (4) Methods of Purification and Distillation of 1,3-BG

Provided herein are methods for purifying a neutral odor bioderived 1,3-BG. For separation of the bioderived 1,3-BG from a microbial organism culture, extraction procedures can be used as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration.

The process for purifying a neutral odor bioderived 1,3-BG can include the steps of culturing a non-naturally occurring microbial organism to produce a bioderived 1,3-BG in a fermentation broth; and subjecting the fermentation broth to one or more of the following procedures: microfiltration, ultrafiltration, nanofiltration, primary ion exchange, evaporation, polishing ion exchange, column distillation, hydrogenation, active-carbon filtration or adsorption, base addition, sodium borohydride (NaBH₄) treatment, and wiped-film evaporation.

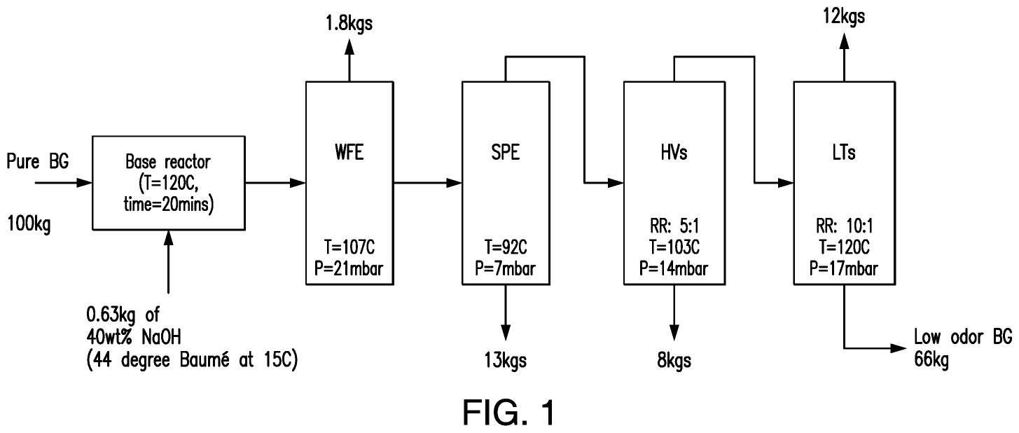

A neutral odor bioderived 1,3-BG can be purified and distilled. The distillation can be carried out with a distillation system to produce a purified bioderived 1,3-BG product, e.g., a neutral odor bioderived 1,3-bioBG or a low odor bioderived 1,3-bioBG. The conventional purification method is a method in which the reaction mixture of 1,3-butylene glycol (e.g., a bioderived 1,3-BG provided herein) produced by liquid phase hydrogen reduction of acetaldol is subjected to removal of alcohols, removal of water, removal of salts and high-boiling materials and removal of high-boiling materials, then in the low-boiling material removal distillation tower (product distillation tower) low-boiling materials are distilled off from the top of the tower and 1,3-butylene glycol (e.g., a neutral odor bioderived 1,3-BG) is obtained as a product from the tower bottom. The addition of a base to bioderived 1,3-butylene glycol having a low content of high-boiling materials and heat-treating the mixture effectively decreases odor-causing materials and gives rise to a neutral odor bioderived 1,3-butylene glycol or a 1,3-butylene glycol having no odor and shows less change with time. The base to be added can include but is not limited to: an alkali metal compound, sodium hydroxide, potassium hydroxide, sodium (bi) carbonate, sodium hydroxide, potassium hydroxide or mixtures thereof. The base may be added in the form of solids as it is or can be added as an aqueous solution for ease of operation and for promoting contact with the target solution. Suitable base reactor and distillation reaction temperatures are provided in FIG. 1. In some embodiments, a distillation reaction temperature ranges from at least about 90 degrees Celsius (° C.) up to about 140° C. In some embodiments, the reaction retention time is at least about 5 minutes up to about 120 minutes. In some embodiments, the reaction retention time is at least about 10 minutes up to about 30 minutes.

From the top of the tower, bioderived 1,3-butylene glycol containing low-boiling materials is distilled off and is charged to the next product distillation tower. The product distillation tower may be a porous plate tower, a foamed bell tower, etc. Preferably, it is a filled tower having a low pressure loss, filled with Sulzer Packing, Melapack™ (both are trade names for products by Sumitomo Heavy Industries, Ltd.), etc. is more suitable. This is because 1,3-butylene glycol is thermally decomposed at 200° C. or higher to affect adversely on odor (See, e.g., U.S. Pat. No. 6,376,725 B1, the contents of which is incorporated herein by reference in its entirety) so that distillation temperature is to be lowered as low as possible. When thermal hysteresis (retention time) to 1,3-butylene glycol is long, similarly influenced. Therefore, the reboiler to be adopted is suitably a thin film evaporator, such as a natural flow-down type thin film evaporator or a forced stirring type thin film evaporator.

The product distillation may depend on the concentration of low-boiling material in the charge stock solution, but when the concentration of low-boiling material in the charge stock solution is 5% or less, it may be one having a theoretical number of daylights of about 10 to 20 (trays). It is preferred that the charge stock solution is fed at a position from the top of the tower to a height 20 to 70% of the height of the tower. At this time, distillation is performed under reduced pressure of 100 torr (133.322 millibars (mbar)) or less, preferably 5 torr to 20 torr (6.66612 mbar-26.6645 mbar), at the top of the tower. For the odor of a bioderived 1,3-butylene glycol to be removed, it is preferred to use a lower distillation temperature. The lower the pressure is, the more suitable. It is desirable to run distillation at a reflux ratio (RR) of at least about 0.5 up to about 15.

The charging into the product distillation tower is carried out by feeding the liquid obtained by concentrating the top of the tower vapor of the dealkalization tower in the condenser. To decrease the calorie for heating the product distillation tower, the top of the tower vapor from the dealkalization tower may be directly fed to the production tower. Bioderived 1,3-butylene glycol product can be obtained from the tower bottom of the product distillation tower. Suitable distillation conditions for generating a low odor bioderived 1,3-bioBG are further described in Example 2.

Alternative distillation protocols known in the art can be used. By way of example only, the treated liquid from the alkali reactor can be fed first into the low-boiling material removal distillation tower where low-boiling materials are distilled off, subsequently the bioderived 1,3-butylene glycol extracted from the gas phase portion in the recover trays or from the tower bottom is distilled or evaporated to remove the base and the resulting high-boiling materials as distillation residues and 1,3-butylene glycol is recovered from the top of the tower or in the midway of concentration trays. The distillate of the above low-boiling materials may further be fed to an additional distillation tower where 1,3-butylene glycol is distilled off to remove high-boiling materials. Also, a portion of the 1,3-butylene glycol after removal of the above low-boiling materials may be recycled to the dealkalization tower. Alternatively, low-boiling materials containing 1,3-butylene glycol may be recycled to the alkali reactor.

A caustic process can occur in an alkali reactor (see, e.g., U.S. Pat. No. 6,376,725, which is incorporated herein by reference in its entirety) or by simply adding a caustic solution, such as, caustic soda, caustic potash, sodium borohydride, and potassium borohydride during deboiling distillation (see, e.g., JP 3369707 B2, which is incorporated herein by reference in its entirety). Accordingly, some embodiments of the disclosure provide a method of obtaining a neutral odor BG comprising one or more (e.g., 2, 3, 4) distillations for use following any caustic (e.g., alkali) process with or without an alkali reactor.

The final deodorized bioderived 1,3-BG can be used in a neutral odor bioderived 1,3-BG product or composition comprising a neutral odor bioderived 1,3-BG product as described herein. The purified bioderived 1,3-BG product (e.g., a neutral odor 1,3-bioBG) can be or can include greater than 90%, 92%, 94%, 96%, 97%, 98%, 99%, 99.5%, 99.7% or 99.9% bioderived 1,3-BG on a weight/weight basis. The recovery of neutral odor bioderived 1,3-BG in the purified bioderived 1,3-BG product can be calculated as a percentage of the amount of neutral odor bioderived 1,3-BG in the purified neutral odor bioderived 1,3-BG product divided by the amount of neutral odor bioderived 1,3-BG or target compound in the crude bioderived 1,3-BG mixture that was purified.

A neutral odor bioderived 1,3-BG provided herein can be obtained by polishing. Polishing involves, e.g., ion exchange chromatography, or contacting with activated carbon. Polishing is a procedure to reduce or remove any remaining salts and/or other impurities in a crude bioderived 1,3-BG mixture, or partially purified bioderived 1,3-BG. The polishing can include contacting the crude bioderived 1,3-BG or partially purified neutral odor bioderived 1,3-BG, with one or a number of materials that can react with or adsorb the impurities in the crude bioderived 1,3-BG mixture or partially purified neutral odor bioderived 1,3-BG. The materials used in the polishing can include ion exchange resins, activated carbon, or adsorbent resins, such as, for example, DOWEX™ 22, DOWEX™ 88, OPTIPORE™ L493, AMBERLITE™ XAD761 or AMBERLITE™ FPX66, or mixtures of these resins, such as a mixture of DOWEX™ 22 and DOWEX™ 88. A neutral odor bioderived 1,3-BG provided herein can be obtained by one or more of fermentation, cell separation, salt separation, evaporation, or a combination thereof. In some embodiments, the process includes fermentation, followed by cell separation, followed by salt separation, followed by evaporation. In some embodiments, fermentation, cell separation, salt separation, and evaporation yield a crude bioderived 1,3-BG mixture or partially purified neutral odor bioderived 1,3-BG that can be fed into a polishing column (e.g., polishing ion-exchange), a dewatering column, or a first distillation column in a process or system. Methods for producing bioderived 1,3-BG in a fermentation broth are described, e.g., in WO 2010/127319 A2 and WO 2011/071682 A1, the entire contents of each of which are incorporated by reference herein.

Centrifugation can be used to provide a crude bioderived 1,3-BG mixture or partially purified neutral odor bioderived 1,3-BG substantially free of solids, including cell mass.

A neutral odor 1,3-bioBG can also be obtained by microfiltration. Microfiltration involves a low-pressure membrane process for separating colloidal and suspended particles in the range of about 0.05-10 microns. Useful configurations include cross-flow filtration using spiral-wound, hollow fiber, or flat sheet (cartridge) microfiltration elements. Microfiltration includes filtering through a membrane having pore sizes from about 0.05 microns to about 10.0 microns. Microfiltration can be used in the clarification of fermentation broth.

A neutral odor 1,3-bioBG can be obtained by ultrafiltration. Ultrafiltration is a selective separation process through a membrane using pressures up to about 145 psi (10 bar). Useful configurations include cross-flow filtration using spiral-wound, hollow fiber, or flat sheet (cartridge) ultrafiltration elements. These elements consist of polymeric or ceramic membranes with a molecular weight cut-off of less than about 200,000 Daltons. Ceramic ultrafiltration membranes are also useful since they have long operating lifetimes of up to or over 10 years. Ceramics have the disadvantage of being much more expensive than polymeric membranes. Ultrafiltration concentrates suspended solids and solutes of molecular weight greater than about 1,000 Daltons. Ultrafiltration includes filtering through a membrane having nominal molecular weight cut-offs (MWCO) from about 1,000 Daltons to about 200,000 Daltons (pore sizes of about 0.005 to 0.1 microns). Using ultrafiltration, the permeate liquid will contain low-molecular-weight organic solutes, such as neutral odor bioderived 1,3-BG, media salts, and water. The captured solids can include, for example, residual cell debris, DNA, and proteins. Diafiltration techniques well known in the art can be used to increase the recovery of neutral odor bioderived 1,3-BG in the ultrafiltration step.

A further filtration procedure called nanofiltration can be used to separate out certain materials by size and charge, including carbohydrates, inorganic and organic salts, residual proteins and other high molecular weight impurities that remain after the previous filtration step. This procedure can allow the recovery of certain salts without prior evaporation of water, for example. Nanofiltration can separate salts, remove color, and provide desalination. In nanofiltration, the permeate liquid generally contains monovalent ions and low-molecular-weight organic compounds as exemplified by neutral odor bioderived 1,3-BG. Nanofiltration includes filtering through a membrane having nominal molecular weight cut-offs (MWCO) from about 100 Daltons to about 2,000 Daltons (pore sizes of about 0.0005 to 0.005 microns). Larger ionic species, including divalent and multivalent ions, and more complex molecules are substantially retained (rejected). Larger non-ionic species, such as carbohydrates are also substantially retained (rejected).

Suitable purification and/or assays to test for the production, chemical purity, or chiral purity of a 1,3-BG (e.g., a neutral odor bioderived 1,3-BG or a low odor bioderived 1,3-BG) can be methods such as HPLC (High Performance Liquid Chromatography), GC-MS (Gas Chromatography-Mass Spectroscopy) and LC-MS (Liquid Chromatography-Mass Spectroscopy), infrared (IR) spectroscopy, and nuclear magnetic resonance (NMR) spectroscopy. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. In some embodiments, the bioderived 1,3-BG has a purity level (e.g., chemical purity) of at least 55%, at least 60%, at least 65%, at least 70%, at least 75%, at least 80%, at least 85%, at least 90%, at least 95%, or at least 99%, e.g., on a weight/weight basis. In some embodiments, the bioderived 1,3-BG has a purity level (e.g., chemical purity) of at least 95%, at least 96%, at least 97%, at least 98%, or at least 99%. In some embodiments, the bioderived 1,3-BG has a purity level (e.g., chemical purity) of at least 99.0%, at least 99.1%, at least 99.2%, at least 99.3%, at least 99.4%, at least 99.5%, at least 99.6%, at least 99.7%, at least 99.8%, or at least 99.9%.

A neutral odor bioderived 1,3-BG can have a higher purity level (e.g., chemical purity) than industrial-grade or cosmetic-grade bio-BG. In some embodiments, the bioderived 1,3-BG has about the same purity level as industrial-grade or cosmetic-grade bio-BG (e.g., a purity level of ±0.5%). In some embodiments, the bioderived 1,3-BG has a lower purity level than industrial-grade or cosmetic-grade bio-BG.

In some embodiments, the bioderived 1,3-BG has a higher purity (e.g., chemical purity) than industrial-grade or cosmetic-grade petro-BG. In some embodiments, the bioderived 1,3-BG has about the same purity level as industrial-grade or cosmetic grade petro-BG (e.g., a purity level of ±0.5%). In some embodiments, the bioderived 1,3-BG has a lower purity level than industrial-grade or cosmetic-grade petro-BG.

### (5) Applications

Provided herein are bioderived 1,3-BG compositions for use as a personal care product, a cosmetic, a therapeutic, a pharmaceutical composition, a nutraceutical, an aromatherapeutic, a fragrance, or a cosmeceutical formulation, wherein said bioderived 1,3-BG does not result in undesirable characteristics or properties when used in a personal care product, a cosmetic, a therapeutic, a pharmaceutic, a nutraceutical, an aromatherapeutic, a fragrance, or a cosmeceutical formulation.

In some embodiments, the bioderived 1,3-BG composition provided herein is a formulated for topical delivery. Topical delivery preferably means delivery to a keratinous layer such as the skin, hair and/or nails, but can also mean delivery to a body lumen lined with epithelial cells, for example the lungs or airways, the gastrointestinal tract, the buccal cavity. The effect may be confined to the surface of the skin or may be within the skin or a combination of both. The topical composition may be applied by, but not limited to, rubbing, or massaging into the keratinous tissue, skin or area of the body to be treated or cared for. In some embodiments, the composition is left on or not removed from the area of the body. In other embodiments, the composition is removed after a period of time, such as, but not limited to, from about 2 minutes to 60 minutes, from about 5 minutes to about 30 minutes, preferably from about 10 minutes to about 20 minutes. The composition may be removed immediately after application. The composition may be administered to the skin, hair, the nails. The composition may be administered in any dose or frequency by any method of topical application.

The composition can be used to treat or care for any disease, disorder or condition of the skin, including but not limited to, psoriasis, dermatitis, allergic dermatitis, eczema, spongiosis, edema, skin cancer, ulcers, acne, scars, cellulitis, elastosis, keratosis, rosacea, varicose veins, inflammatory disorders.

Further provided herein are bioderived 1,3-BG compositions for use in the treatment and/or care of conditions of the skin. Further provided herein are bioderived 1,3-BG compositions for use in slowing or inhibiting, or preventing ageing of human skin. In some embodiments, the bioderived 1,3-BG composition provided herein is formulated as an anti-ageing solution, cream, or serum. The topical composition may be used to for treating or caring for visible signs of aging including but not limited to wrinkles, stretch marks and dark circles, dryness, fine lines, age spots, red blotches, sagging skin, and conditions caused by sun exposure including sunburn, stress, pollution and/diet. The topical composition may also be used for delaying, slowing or inhibiting the skins or the onset of aging.

Further provided herein are bioderived 1,3-BG compositions for use as flavoring agent, a food additive, or a drink additive.

### EXAMPLES

The following examples illustrate specific aspects of the instant description. The examples should not be construed as limiting, as the example merely provides specific understanding and practice of the embodiments and its various aspects.

### Example 1: Methods of Producing Bioderived 1,3-BG

Bioderived 1,3-butylene glycol was produced by genetically modified, non-naturally occurring microbial organisms.

Microbial strains were engineered to have a functional 1,3-butanediol synthesis pathway that converts alanine to 1,3-BG. Other pathway precursors and intermediates were used to produce bioderived 1,3-BG including an acetoacetyl-CoA intermediate and/or a 4-hydroxybutyryl-CoA precursor The expression of the pathway genes is corroborated using methods for determining polypeptide expression and enzymatic activity, including, Northern blots, PCR amplification of mRNA, and immunoblotting. Enzymatic activities of the expressed enzymes were confirmed using assays specific for the individual activities. The ability of the engineered microbial organism strain to produce 1,3-butanediol were confirmed using HPLC, gas chromatography-mass spectrometry (GC/MS) or liquid chromatography-mass spectrometry (LCMS).

The microbial organisms were assessed to determine whether any of the exogenous genes were expressed at a rate limiting level. Expression was increased for enzymes expressed at low levels that were limiting the flux through the pathway by, for example, introduction of additional gene copy numbers. To generate better producers, metabolic modeling was is utilized to optimize growth conditions. Modeling was also used to design gene knockouts that additionally optimize utilization of the pathway.

For large-scale production of bioderived 1,3-butanediol, a fermentation broth enriched in bioderived 1,3-BG was produced using a protocol as described, e.g., in WO 2010/127319 A2 and WO 2011/071682 A1, the entire contents of each of which are incorporated by reference herein. In brief, an exemplary or a preferred microbial route to a 1,3-bio-BG is described in WIPO patent publication WO2010127319A2, see especially routes comprising a 3-hydroxybutyryl-CoA dehydrogenase, as for example the pathway from acetoacetyl-CoA to 1,3-butanediol fermentations were performed in either a batch, fed-batch or continuous manner. Anaerobic conditions were maintained by first sparging the medium with nitrogen and then sealing culture vessel (e.g., flasks sealed with a septum and crimp-cap). Microaerobic conditions were utilized by providing a small hole for limited aeration. The pH of the medium is maintained at a pH of 7 by addition of an acid, such as H₂SO₄. The growth rate was determined by measuring optical density using a spectrophotometer (600 nm), and the glucose uptake rate by monitoring carbon source depletion over time. Byproducts such as undesirable alcohols, organic acids, and residual glucose were quantified by HPLC (Shimadzu) with an HPX-O 87 column (BioRad®), using a refractive index detector for glucose and alcohols, and a UV detector for organic acids.

Bioderived 1,3-BG was subsequently purified from the fermentation broth using a sequence of (1) microfiltration, (2) nanofiltration, (3) ion-exchange chromatography, (4) evaporation of water, and (5) polishing ion-exchange to produce a crude mix containing bioderived 1,3-BG. The crude mix was then fed into a dewatering distillation column to produce a 1,3-BG-containing product stream that was fed into a 2 L batch distillation column to produce a bioderived 1,3-BG product. The batch distillation column was a randomly packed column of 1″ diameter, about 2 ft tall, and had a condenser and reflux control attached directly on top of the column.

Further improvements in the purity and odor of bioderived 1,3-BG was achieved using a continuous distillation process using the protocol described, e.g., in US2021/0101855A1, the contents of which is incorporated by reference in its entirety. Comparative purity evaluations of 1,3-bio-BG and petro-BG samples were conducted using gas-chromatography/mass spectrometry (GC-MS) analysis further described in Examples 3-4.

### Example 2: Deodorization of Bioderived 1,3-Butylene Glycol

Bg Deodorization Method to Produce Neutral Odor Sample 1,3-bioBg-A

Bioderived 1,3-butylene glycol (bioBG) was produced and deodorized using a caustic treatment followed by distillation described here to produce a bioBG product 1 of the disclosure (1,3-bioBG-A). The block flow diagram which describes the process implemented to produce 800 kgs of deodorized bioBG. See, FIG. 1.

Odorous bioBG was treated with a caustic solution (˜40 wt %-˜50 wt % NaOH), such that the final concentration of NaOH in the reaction solution was 0.25 wt %. This mixture was then reacted in a tank vessel (base reactor) at 120° C. for 20 mins while stirring and as a result, impurities, such as ketones and aldehydes, convert to other less volatile materials. Alternatively, the continuous flow reactor with heat exchanger could be used to carry out the reaction.

The reaction product was then fed into a WFE (wiped film evaporator) for degassing and water removal. This helps in reaching low vacuum in subsequent steps. The bottom stream from the WFE was then fed into a SPE (short pass evaporator) for base removal along with some heavies. This step was typically run at a relatively milder temperature (˜90° C.) and a short residence time (in seconds) to avoid formation of degradation products.

The lights vapor stream from the SPE was then condensed and fed into the 1st distillation column, equipped with a wiped film evaporator (WFE) as a reboiler, where residual heavies are removed as bottoms under vacuum (10-20 torr) at 100° C.-110° C. The tops from this step was sent to a 2^{nd} distillation column of the same size where the lights components were distilled off and a low odor BG (1,3-bioBG-A) was drawn from the bottom. The pressure in this column was low, 10 torr-20 torr at 120° C.; however, had a higher reflux ratio than the heavies removal step for better separation.

BG Deodorization Method to Produce Neutral Odor Sample 1,3-bioBG-B

Crude high odor bioderived 1,3-butylene glycol (bioBG) was produced and deodorized using the caustic distillation process described here to produce a bioBG Product 2 (1,3-bioBG-B).

### Crude Analysis

The crude, BIO-BG Natural Butylene Glycol (D2413) was received in 4×500 mL glass bottles. The odor of the crude was noticeable. This high odor bioBG was deodorized as described here in Table 3.

TABLE 3

| Deordorization tests and results. | |
|---|---|
| TEST | RESULT |
| Water | 0.04% |
| 1,3-butanediol | 99.6781%* |
| GC Lights | 0.1456%* |
| GC Heavies | 0.1336%* |
| Acidity (as acetic acid) | 0.0027% |

*GC results normalized for water and acidity

### Distillation Details

1,3-Butanediol (1,731.8 g) (bioBG) was charged to a 2 L 3 necked flask and placed under an 24 theoretical plate column fitted with a poly(tetrafluorethylene) (PTFE) (0.25 mm ID) nitrogen sparge. The temperature was monitored using a PTFE thermocouple in the base and a stainless steel thermocouple in the side stream and overheads which were connected to a RS52 2channel K handheld thermometer. After 18 theoretical plates, a side stream was fitted and was connected to a Perkin triangle and 250 ml round bottom flask. A reflux controller and Perkin triangle fitted with a 250 ml round bottom flask were used as overheads. A vacuum of 20 mmHg was applied to the system using a Javac DS75 high vacuum pump. The system was brought to a boil and held at a total reflux for ˜15 minutes. After this, because the temperatures at the side stream and at the column top were similar, the top reflux ratio was set to 1:1 to remove any light boilers for ˜45 minutes. The side stream was then set to take off 1,3-butanediol at 1:1 and the reflux ratio adjusted to ˜10:1. The purity and the odor of the side stream was unacceptable, so the side stream take off was stopped while more lights were removed from the column top at a reflux ratio of ˜15:1 or ˜10:1. On start up the following day, the side stream was set to take off 1,3-butanediol at 1:1 and the top reflux ratio set to take off at 5:1. The reflux ratio at the column top was varied slightly through the distillation to determine if the odor and purity of the side stream improved with an increased reflux ratio. The rest of the distillation was run with a top reflux ratio of ˜5:1 and a side stream take off of 1:1. Towards the end of the distillation, the GC heavies in the side stream began to increase and knocked the purity of the side stream below the specification of a 99.5% minimum.

### Distillation Run Sheet

The middle temperature (MT) read low during the run sheet until Apr. 7, 2021 (07/04/2021), which was due to a lack of lagging on the side stream portion of the column.

TABLE 4
Distillation Run Sheet.

| | | | | | | | | |
|---|---|---|---|---|---|---|---|---|
| | BT | MT | TT | Middle | Top | Pressure | | Observations/ |
| Time | (° C.) | (° C.) | (° C.) | RR | RR | (mmHg) | Fraction | Comments |

30 Mar. 2021

| | | | | | | | | |
|---|---|---|---|---|---|---|---|---|
| 08:30 | 16.9 | 17.9 | 18.3 | Total | Total | 21 | | Heat on. |
| 09:33 | 93.1 | 18.3 | 18.4 | Total | Total | 20 | | |
| 10:24 | 121.5 | 85.4 | 19.4 | Total | Total | 20 | | |
| 10:54 | 122.8 | 108.7 | 86.0 | Total | Total | 20 | | |
| 11:03 | 123.0 | 111.3 | 118.9 | Total | Total | 20 | | |
| 11:14 | 122.6 | 110.9 | 119.7 | Total | Total | 20 | | TR → 2:1 |
| 12:00 | 121.8 | 114.2 | 119.3 | Total | 1:1 | 20 | T1 | |
| | | | | | | | (22.9 g) | |
| 12:41 | 122.1 | 113.9 | 119.2 | Total | 2:1 | 20 | T2 | SS flush |
| | | | | | | | (24.3 g) | |
| 12:49 | 122.4 | 113.9 | 119.0 | Total | 2:1 | 20 | SS1 | MR → 2:1 |
| | | | | | | | (9.8 g) | |
| 13:34 | 121.7 | 113.2 | 118.5 | 2:1 | 2:1 | 20 | SS2 | TR → 10:1 |
| | | | | | | | (16.5 g) | |
| | | | | | | | T3 | |
| | | | | | | | (28.0 g) | |
| 14:23 | 121.2 | 112.4 | 117.1 | 2:1 | 13:1 | 19 | SS3 | MR → Total |
| | | | | | | | (13.0 g) | Take more tops |
| 14:35 | 120.7 | 112.8 | 117.4 | Total | 10:1 | 19 | T4 | |
| | | | | | | | (13.1 g) | |
| 15:17 | 120.4 | 112.1 | 116.8 | Total | 6:1 | 19 | | TR → 3:1 |
| 15:56 | 120.4 | 113.1 | 117.2 | Total | 3:1 | 19 | T5 | Heat off. |
| | | | | | | | (26.7 g) | |

31 Mar. 2021

| | | | | | | | | |
|---|---|---|---|---|---|---|---|---|
| 07:54 | 25.3 | 36.3 | 30.6 | Total | Total | | | Heat & vacuum on. |
| 08:49 | 121.6 | 113.6 | 20.6 | Total | Total | 20 | | |
| 09:02 | 122.2 | 116.7 | 119.4 | Total | Total | 20 | | Refluxing. |
| 09:26 | 122.4 | 117.4 | 119.6 | Total | Total | 20 | | TR → 5:1 |
| 10:26 | 123.6 | 119.2 | 120.9 | Total | 2:1 | 20 | SS4 | SS flush (SS4) |
| | | | | | | | (5.1 g) | MR → 2:1 |
| 11:15 | 123.1 | 117.8 | 119.7 | 1:1 | 5:1 | 20 | SS5 | |
| | | | | | | | (16.9 g) | |
| 12:03 | 128.3 | 123.6 | 119.6 | 1:1 | 5:1 | 20 | SS6 | |
| | | | | | | | (13.4 g) | |
| | | | | | | | T6 | |
| | | | | | | | (58.3 g) | |
| 13:02 | 126.2 | 120.9 | 119.6 | 1:1 | 3:1 | 20 | SS7 | |
| | | | | | | | (25.2 g) | |
| 13:32 | 124.3 | 119.2 | 119.5 | 1:1 | 2:1 | 20 | T7 | |
| | | | | | | | (40.1 g) | |
| 13:47 | 123.9 | 118.8 | 119.7 | 1:1 | 3:1 | 20 | SS8 | MR → Total |
| | | | | | | | (12.4 g) | |
| | | | | | | | | TR → 2:1 |
| 14:10 | 127.4 | 121.8 | 120.0 | Total | 2:1 | 20 | T8 | |
| | | | | | | | (22.2 g) | |
| 14:46 | 126.2 | 121.3 | 120.1 | Total | 2:1 | 20 | T9 | |
| | | | | | | | (23.2 g) | |
| 15:36 | 127.6 | 122.3 | 120.0 | Total | 2:1 | 20 | T10 | |
| | | | | | | | (33.8 g) | |
| 16:02 | 126.2 | 121.1 | 120.2 | Total | 2:1 | 20 | | MR → 1:1 |
| 16:35 | 127.7 | 121.9 | 120.0 | 1:1 | 2:1 | 20 | SS9 | Odour better, purity |
| | | | | | | | (24.9 g) | 99.6% |
| | | | | | | | | TR → 10:1 |
| 17:08 | 127.8 | 122.2 | 119.4 | 1:1 | 10:1 | 22 | SS10 | |
| | | | | | | | (34.4 g) | |
| 17:31 | 124.2 | 118.2 | 119.6 | 1:1 | 10:1 | 22 | SS11 | Heat off. |
| | | | | | | | (30.5 g) | |
| | | | | | | | T11 | |
| | | | | | | | (50.8 g) | |

1 Apr. 2021

BS1 (46.2 g)
Taken for GC, odor
analysis and potential
chemical treatment
for odor removal.

6 Apr. 2021

| | | | | | | | | |
|---|---|---|---|---|---|---|---|---|
| 09:07 | 13.9 | 16.1 | 16.4 | Total | Total | | | Heat & vacuum on. |
| 10:09 | 124.1 | 115.9 | 18.9 | Total | Total | 20 | | |
| 10:19 | 124.4 | 117.2 | 121.7 | Total | Total | 20 | | Refluxing. |
| 10:37 | 127.1 | 119.9 | 121.6 | Total | Total | 20 | | TR → 10:1 |
| 11:06 | 131.6 | 123.6 | 121.7 | Total | 10:1 | 20 | | MR → 1:1 |
| 11:46 | 134.1 | 127.2 | 121.7 | 1:1 | 8:1 | 20 | SS12 | |
| | | | | | | | (27.4 g) | |
| 12:17 | 125.6 | 118.7 | 121.5 | 1:1 | 5:1 | 20 | SS13 | |
| | | | | | | | (36.7 g) | |
| 13:39 | 125.0 | 117.8 | 121.4 | 1:1 | 5:1 | 20 | SS14 | |
| | | | | | | | (87.2 g) | |
| 14:39 | 125.5 | 118.5 | 121.5 | 1:1 | 5:1 | 20 | SS15 | |
| | | | | | | | (106.6 g) | |
| | | | | | | | T12 | |
| | | | | | | | (50.0 g) | |
| 16:02 | 124.7 | 117.6 | 121.2 | 1:1 | 5:1 | 20 | SS16 | Heat off. |
| | | | | | | | (149.1 g) | |

7 Apr. 2021

| | | | | | | | | |
|---|---|---|---|---|---|---|---|---|
| 09:28 | 14.4 | 17.2 | 17.7 | Total | Total | 20 | | Heat on. |
| 10:46 | 122.5 | 115.2 | 119.7 | Total | Total | 20 | | Refluxing. |
| 10:59 | 122.8 | 115.9 | 120.3 | Total | Total | 20 | | TR → 10:1 |
| | | | | | | | | MR → 1:1 |
| 11:25 | 122.8 | 115.9 | 120.3 | 1:1 | 10:1 | 20 | SS17 | Lagging added to SS. |
| | | | | | | | (34.3 g) | |
| 12:18 | 127.2 | 123.1 | 119.7 | 1:1 | 10:1 | 20 | SS18 | |
| | | | | | | | (100.4 g) | |
| 13:03 | 127.2 | 122.3 | 119.7 | 1:1 | 5:1 | 20 | SS19 | MR → Total |
| | | | | | | | (114.2 g) | SS19 purity 99.2% |
| 13:46 | 126.8 | 122.3 | 119.7 | Total | 5:1 | 20 | T13 | MR → 1:1 |
| | | | | | | | (42.4 g) | |
| 15:17 | 124.9 | 119.6 | 119.6 | 1:1 | 5:1 | 20 | SS20 | Heat off. |
| | | | | | | | (204.1 g) | |

BT—bottom temperature;
MT—middle temperature;
TT—top temperature;
RR—reflux ratio;
SS—side stream;
T—tops

### Fraction Analysis as GC Area (%)

1,3-Butanediol is quite viscous and significant variation was seen in the repeat injection of the crude sample. For this reason, all GC analysis was performed as a 50:50 mixture with water.

Analysis of the crude and composite was performed neat. The syringe was thoroughly washed with water between each run.

TABLE 5
Analysis of crude and composite fractions.

| Fraction | Mass (g) | % GC Lights | % 1,3-Butanediol | % GC Heavies | Odour |
|---|---|---|---|---|---|
| Crude * | 1731.8 | 0.0521 | 98.5535 | 1.3517 | |
| SS1 | 9.8 | 0.1769 | 99.4564 | 0.3667 | smelly |
| SS2 | 16.5 | 0.0129 | 99.1592 | 0.8279 | smelly |
| SS3 | 13.0 | 0.0106 | 99.3232 | 0.6662 | smelly |
| SS4 | 5.1 | 0.0163 | 99.6661 | 0.3176 | smelly |
| SS5 | 16.9 | 0.0061 | 99.6748 | 0.3191 | smelly |
| SS6 | 13.4 | 0.0046 | 99.1464 | 0.8490 | smelly |
| SS7 | 25.2 | 0.0131 | 98.3314 | 1.6555 | smelly |
| SS8 | 12.4 | 0.0088 | 99.1968 | 0.7944 | milder |
| SS9 | 24.9 | 0.0065 | 99.6453 | 0.3482 | milder |
| SS10 | 34.4 | 0.0048 | 99.5572 | 0.4380 | mild |
| SS11 | 30.5 | 0.0044 | 99.6370 | 0.3586 | mild |
| SS12 | 27.4 | 0.0097 | 99.4827 | 0.5076 | mild |
| SS13 | 36.7 | 0.0026 | 99.5393 | 0.4581 | very low |
| SS14 | 87.2 | 0.0021 | 99.7069 | 0.2910 | mild |
| SS15 | 106.6 | 0.0028 | 99.5918 | 0.4054 | mild |
| SS16 | 149.1 | 0.0013 | 99.6303 | 0.3684 | mild |
| SS17 | 34.3 | 0.0043 | 99.7037 | 0.2920 | mild |
| SS18 | 100.4 | 0.0025 | 99.2659 | 0.7316 | slightly fruity? |
| SS19 | 114.2 | 0.0027 | 99.3554 | 0.6419 | mild |
| SS20 | 204.1 | 0.0011 | 99.4540 | 0.5449 | mild |
| T1 | 22.9 | 0.9946 | 96.3821 | 2.6233 | |
| T2 | 24.3 | 0.3910 | 97.6079 | 2.0011 | |
| T3 | 28.0 | 0.1349 | 97.0450 | 2.8201 | |
| T4 | 13.1 | 0.1222 | 98.9421 | 0.9357 | |
| T5 | 26.7 | 0.0348 | 98.8430 | 1.1222 | |
| T6 | 58.3 | 0.0167 | 99.2596 | 0.7237 | |
| T7 | 40.1 | 0.0050 | 98.9969 | 0.9981 | |
| T8 | 22.2 | 0.0035 | 99.4025 | 0.5940 | |
| T9 | 23.2 | 0.0044 | 99.2474 | 0.7482 | |
| T10 | 33.8 | 0.0045 | 99.2555 | 0.7400 | |
| T11 | 50.8 | 0.0004 | 99.7298 | 0.2698 | |
| T12 | 50.0 | 0.0080 | 99.6148 | 0.3772 | |
| T13 | 42.4 | 0.0059 | 99.7772 | 0.2169 | |
| T14 | 20.8 | 0.0007 | 99.8123 | 0.1870 | |
| BS1 | 46.2 | | | | |
| Residues | 112.2 | n/d | 99.7989 | 0.2011 | |

* GC results normalized for water and acidity.

### Residues

The residues (112.2 g) consisted of a pale yellow colored liquid containing no solids. The residues had no significant odor.

The walls of the flask were clean once the residues were decanted, a cold water wash was used to clean the flask successfully.

### Composite Sample

A composite sample (SS 9-20, 949.8 g) was prepared and analyzed as provided in Table 6.

TABLE 6
SS 9-20 analysis.

| Test | Result | Specification |
|---|---|---|
| Water | 0.02% | |
| 1,3-Butanediol | 99.9238% | 99.5% min. |
| GC Lights | 0.0150% * | |
| GC Heavies | 0.0392% * | |
| Color | 0 Hazen | 10 Hazen max. |
| Appearance | Clear Colorless Liquid | Clear Colorless Liquid |
| SG (at 20°° C.) | 1.005 | 1.004 to 1.006 |
| Odor | Odorless/Low Odor | Odorless |
| Free acid (as acetic acid) | 0.002% | 0.005% max. |

* GC results normalized for water and acidity.

### Mass Balance

TABLE 7
Mass balance.

| | Weight (g) | Percentage (%) |
|---|---|---|
| Total Mass In | 1731.8 | 100.00 |
| Spec 13BD (SS) | 949.8 | 54.84 |
| Non spec 13BD (SS) | 112.3 | 6.48 |
| Lights/Tops | 456.6 | 26.37 |
| Residues | 112.2 | 6.48 |
| Base Samples | 46.2 | 2.67 |
| Losses | 54.7 | 3.16 |

### CONCLUSION

The odor of the lights fractions all had a significant odor. The odor of the early side stream fractions also had a significant odor which eventually reduced to an acceptable level. A lot of the charge was taken as tops and off-spec side stream, this resulted in a poor yield of ˜55%. Throughout the distillation, the reflux ratio at the column top had to be altered to keep the side stream purity on spec.

The yield of the distillation can be improved upon if repeated by increasing the top reflux ratio to 15:1, on the plant there is also the opportunity to recycle inters type material which would increase the yield. To improve the yield in the lab, the side stream take off should not start until the purity in the tops fractions was >99%, which can occur by slowly removing light boilers at a higher reflux ratio of ˜15:1.

### Corrosion Studies

A 321 corrosion coupon was placed in the flask during the distillation of 1,3-butanediol. The corrosion rate was deemed acceptable. The coupon showed no weight loss.

### Other Work

BS1 was taken, split, and treated with Venpure (0.7%), NaOH (10%) and NaBH₄ (0.5%) to determine whether any improvement in odor was possible. The samples were mixed and placed in the oven for a short period of time before their odor was checked using the hot water test (20 mL hot water+1 mL sample). The general opinion was that there was no odor improvement observed.

It was difficult to conclude whether any of these treatment options would provide a better odor without running through the entire distillation process.

### Analytical Details

The following equipment and conditions were used to analyze the distillation fractions provided in Table 8.

TABLE 8
Equipment and conditions for analysis of distillation fractions.

| | |
|---|---|
| Column Type/Dimensions | HP-1; 30 m × 0.32 mm |
| | (film thickness 1.0 μl) |
| Carrier Gas/Pressure | Hydrogen/ 5.0 psig |
| Injector/Detector Temps | 250° C./270° C. |
| Sample size | 0.1 μl |
| Temp. Programme | 80° C. to 260° C. at 20° C./min |
| | (iso 11 mins) |
| Total Run Time | 20 Minutes |
| Split Ratio | 20:1 |
| GC | GC-3 |
| Method File | 1,2-Pentanediol |
| Method Name | CC5_HP1 |

The following equipment and conditions were used to analyze the spec composite and crude samples provided in Table 9.

TABLE 9
Equipment and conditions for analysis of composite
and crude samples.

| | |
|---|---|
| Column Type/Dimensions | CP7834: CP-Sil 24CB; 30 m × 0.32 mm |
| | (film thickness 0.5 μl) |
| Carrier Gas/Pressure | Hydrogen/ 5.0 psig |
| Injector/Detector Temps | 250° C./270° C. |
| Sample size | 1.0 μl |
| Sample preparation | 10 drops sample: 1 mL HPLC |
| | methanol, mixed well before injection |
| Temp. Programme | 80° C. to 240° C. at 20° C./min |
| | (iso 11 mins) |
| Total Run Time | 20 Minutes |
| Split Ratio | Splitless |
| Integration Notes | Lock out methanol peak |
| GC | GC-3 |
| Method File | 1,3-Butylene Glycol |
| Method Name | CIQC 210328 |

### Equipment

This distillation work used the ‘new’ side stream column which was installed in 2020. The ‘new’ and ‘old’ side stream columns were set up very similarly. Both side stream columns had an 18-plate column fitted with a side stream and a 6-plate column on top of that.

The differences between the columns are that the ‘old’ side stream used knit mesh packing and the ‘new’ side stream used Sulzer packing. The ‘old’ side stream also had column heating fitted to the lower 18-plate and upper 6-plate column. The ‘new’ side stream only had column heating fitted to the lower 18-plate column. For this reason, the boil up achieved in the upper column of the ‘new’ side stream was not as good as the ‘old’ side stream, meaning a slower reflux and distillation.

The ‘new’ side stream column was then fitted with temporary column heating on the upper column to help minimize differences between the columns.

### Specifications

The specifications of a number of suppliers were compared and an initial specification was chosen for the work described here.

The table below details the specifications for 1,3-butanediol for various suppliers and the target specification aimed to meet. The market leader is Diacel. The selected target specification was the same as Godavari's but with a tighter acidity spec of 0.005% max. as acetic acid.

TABLE 10
Specifications of 1,3-butanediol for various suppliers.

| | | | | Nihon | |
|---|---|---|---|---|---|
| | Diacel | KH NeoChem | Godavari | Koken | Target Spec |
| Appearance | Clear | Clear Colorless | Clear | Colorless | Clear Colorless |
| | Colorless | Liquid Almost | Colorless | Liquid | Liquid |
| | | Odourless | Liquid | | |
| Color | 15 Pt-Co max. | 10 APHA max. | 10 Hazen max. | | 10 Hazen max. |
| SG (20/20° C.) | 1.004 to | 1.004 to 1.007 | 1.004 to 1.006 | | 1.004 to 1.006 |
| | 1.007 | | | | |
| Purity | | 99.5% min. | 99.50% min. | 99.50% | 99.50% min. |
| | | | | min. | |
| Water Content | 0.5% max. | 0.5% max. | | | |
| Distillation, | 203 min. | 200 min. | | | |
| Initial boiling | | | | | |
| point | | | | | |
| Distillation, Dry | 209 max. | 215 max. | | | |
| point | | | | | |
| Free acid (as | 0.005% max. | 0.005% max. | 0.010% max. | | 0.005% max. |
| acetic acid) | | | | | |
| Distillate | | 95% min. | | | |
| pH | | Neutral | | | |
| Odor | | | Normal | Odorless | Odorless |

### SUMMARY

Crude 1,3 Butanediol was added to a laboratory distillation system equipped with a reflux head and side stream take off. A vacuum of 20 mmHg was then applied. The system was brought to boil and held at total reflux for ˜15 minutes. At this point, the temperatures at the side stream and the column top were similar. The top reflux ratio was set to 1:1 to and light boilers removed for ˜45 minutes. After this time, the side stream was set to take off 1,3-butanediol at 1:1 and the reflux ratio was adjusted to ˜10:1. Under these conditions, the purity and the odor of the side stream were deemed unacceptable, and the side stream take off was stopped. Further lights were removed from the column top at a reflux ratio of ˜15:1 for the remainder of the day before being allowed to cool overnight. The column was restarted the next day under similar conditions. The side stream was set to take off 1,3-butanediol at 1:1 and the top reflux ratio was set to take off at 5:1. Small variations in the reflux ratio were made and the impact on odor and purity were observed. Generally, the odor and purity of the side stream improved with an increased reflux ratio. The rest of the distillation was run with a top reflux ratio of ˜5:1 and a side stream take off of 1:1. Towards the end of the distillation, the GC heavies in the side stream began to increase and knocked the purity of the side stream below the specification of a 99.5% minimum.

High odor bioBG was treated with a caustic solution (˜40 wt %-50 wt % NaOH), such that the final concentration of NaOH in solution was 0.5 wt %. This mixture of caustic solution in high odor bioBG was then reacted in a tank vessel or caustic reactor at 60° C. for 6 hours while stirring.

The reaction product was then fed into a distillation column where, the unreacted caustic and insoluble reaction product were removed as a bottoms waste; whereas, pure BG, referred to as a lights fraction, was distilled from the top of the column. Further distillation can be completed if required.

### Example 3: GC/MS and GC/O Methods for Characterizing Bioderived 1,3-BG

Several samples of bioderived 1,3-BG and cosmetic grade petro-BG products were characterized by gas chromatography/mass spectrometry (GC/MS). Table 11 lists the samples tested by GC/MS.

TABLE 11
Sample List.

| Sample Name | Contents and Description | FIG. # | Relative Odor Quality |
|---|---|---|---|
| 1,3-bioBG-A | >99.7 wt % neutral odor bioderived | 3 | Good, neutral odor |
| | 1,3-butylene glycol eith additional | | |
| | purification and distillation described in | | |
| | Example 2 | | |
| 1,3-bioBG-B | >99.7 wt % neutral odor | 3 | Good, neutral odor |
| | bioderived 1,3-butylene | | |
| | glycol with additional | | |
| | purification and distillation | | |
| | described in Example 2 | | |
| 1,3-bioBG-C | >99.7 wt % bioderived 1,3- | 2 and 4 | Medium |
| | butylene glycol | | |
| 1,3-bioBG-D | >99.7 wt % bioderived 1,3- | 4 | Medium |
| | butylene glycol | | |
| 1,3-bioBG-E | >99.7 wt % odorous | 4 | Medium |
| | bioderived 1,3-butylene | | |
| | glycol | | |
| 1,3-bioBG-F | >99.7 wt % bioderived 1,3- | 5 | Medium |
| | butylene glycol | | |
| 1,3-bioBG-G | >99.7 wt % bioderived 1,3- | 5 | Medium |
| | butylene glycol that has | | |
| | undergone odor evaluations | | |
| | by a panel of trained | | |
| | professionals | | |
| 1,3-bioBG-H | >99.7 wt % bioderived 1,3- | 5 | Medium |
| | butylene glycol with high | | |
| | color characteristic | | |
| 1,3-bioBG-I | >99.7 wt % bioderived 1,3- | 5 | Medium |
| | butylene glycol | | |
| 1,3-bioBG-J | >99.7 wt % bioderived 1,3- | Data not | Bad |
| | butylene glycol from 2017 | shown | |
| Petro-1,3-BG-A | >99.7 wt % petro-1,3-butylene | 3 | Good |
| | glycol Cosmetic Grade | | |
| Petro-1,3-BG-B | >99.7 wt % petro-1,3-butylene | 3 | Good |
| | glycol cosmetic grade circa | | |
| | 2021 | | |
| Petro-1,3-BG-C | >99.7 wt % petro-1,3-butylene | 4 | Medium |
| | glycol cosmetic grade circa | | |
| | 2020 | | |

Solvent extraction of the sample was performed using water and dichloromethane (DCM). For each sample assayed by GC/MS, 10 ml of sample was extracted and mixed with 10 milliliters (mL) of water and 10 mL of DCM. The sample and solvents were mixed vigorously. The DCM layer was collected and concentrated to 200 μL under nitrogen gas. The concentrated volatile extract and 1 microliter of the sample were injected into the GC/MS to determine the constituents of each sample in Table 11 (FIG. 2).

GC/MS/O analyses were performed using an AGILENT® 6890N Gas Chromatograph (Agilent Technologies, Santa Clara, CA) equipped with a DB-5 ms column (30 m×0.25 mm ID×0.25 μm film thickness; Agilent Technologies) and coupled to an AGILENT® 5973 Mass Selective Detector (Agilent Technologies) and a Gerstel sniffing port (ODP2; Gerstel, Mülheim ander Ruhr, Germany). The effluent was split 1:1 after column separation to the MSD and sniffing port. Nitrogen was bubbled through distilled water and purged at the end of the heated (250° C.) sniffing arm to reduce nasal dehydration. Helium was used as a carrier at a constant flow of 1.6 mL/min. The GC oven temperature program was as follows: initial temperature was held at 40° C. for 1 min and then ramped to 240° C. at a rate of 8° C./min and held for 5 min. The MSD conditions were as follows: capillary transfer line was set at 250° C.; quadrupole temperature, 150° C.; electron ionization (EI) energy, 70 eV; EI source temperature, 230° C.; mass scanning range, 35-300 m/z. Injection volume was 1 μL in splitless mode, and inlet temperature was held at 250° C.

Each extract was evaluated by 2 panelists experienced in GC/MS/O analysis protocols, who were directed to record the retention time and perceived odor descriptor of detected odorants. Compounds in odor-active regions detected by both panelists were identified through further analysis of the extracts using an Agilent 7890A GC (Agilent Technologies, Santa Clara, CA) equipped with a DB-5 ms Ultra Inert column (60 m×0.250 mm×0.25 μm, Agilent Technologies) and coupled with an Agilent 7250 Quadrupole Time-of-Flight Mass Spectrometer (Agilent Technologies). Injection volume was 2 μL in splitless mode, and inlet temperature was held at 250° C. Helium was used as a carrier at a constant flow of 1.2 mL/min. The GC oven temperature program was as follows: initial temperature was held at 40° C. for 1 min and then ramped to 240° C. at a rate of 6° C./min and held for 10 min. MS conditions used were as follows: capillary transfer line temperature, 250° C.; quadrupole temperature, 150° C.; source temperature, 230° C.; ionization mode, electron ionization (EI); electron energy, 70 eV; emission current, 11.9 uA; mass scanning range, 35-500 m/z. Compound identification was performed with Agilent Unknowns Analysis v10.0 using the SureMass algorithm for data processing and by injection of pure compound standards were possible. Library match scores for tentatively identified compounds are reported on a 100-point scale. The linear retention index (LRI) of compounds on a DB-5 column were calculated with injection of n-alkanes from C7-C30 in the same condition as the samples. Relative concentrations of compounds in test versus control samples were calculated using internal standard normalized peak areas.

### Example 4: Analysis of Aroma Compounds Using Gas Chromatography/Mass Spectrometry/Olfactometry (GC/MS/O) and Gas Chromatography/Quadrupole Time-of-Flight Mass Spectrometry (GC/QTOF-MS)

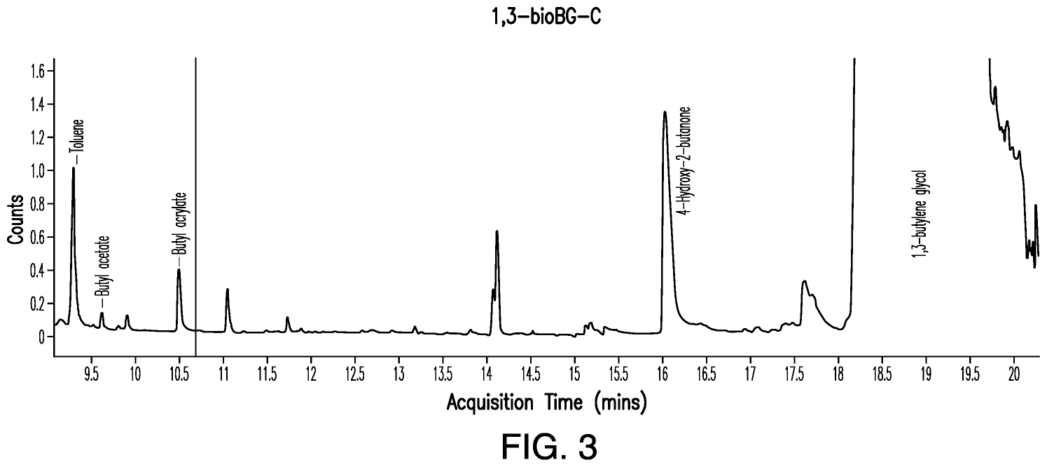

Test and control samples were evaluated by GC/MS as described in Example 3. FIG. 3 shows a representative GC/MS chromatograph of 1,3-bioBG-C. 1,3-bioBG-C exhibits the following peaks set out in Table 12.

TABLE 12
1,3-bioBG-C Counts % vs. Acquisition Time Peaks

| Counts % Acquisition Time(s) | Intensity | Chemical |
|---|---|---|
| 9.7 | 1.0 | toluene |
| 9.6 | <0.2 | butyl acetate |
| 10.5 | 0.4 | butyl acrylate |
| 14 | 0.6 | unknown |
| 16 | 1.2 | 4-hydroxy-2-butanone |
| 18-20 | >1.6 | 1,3-butylene glycol |

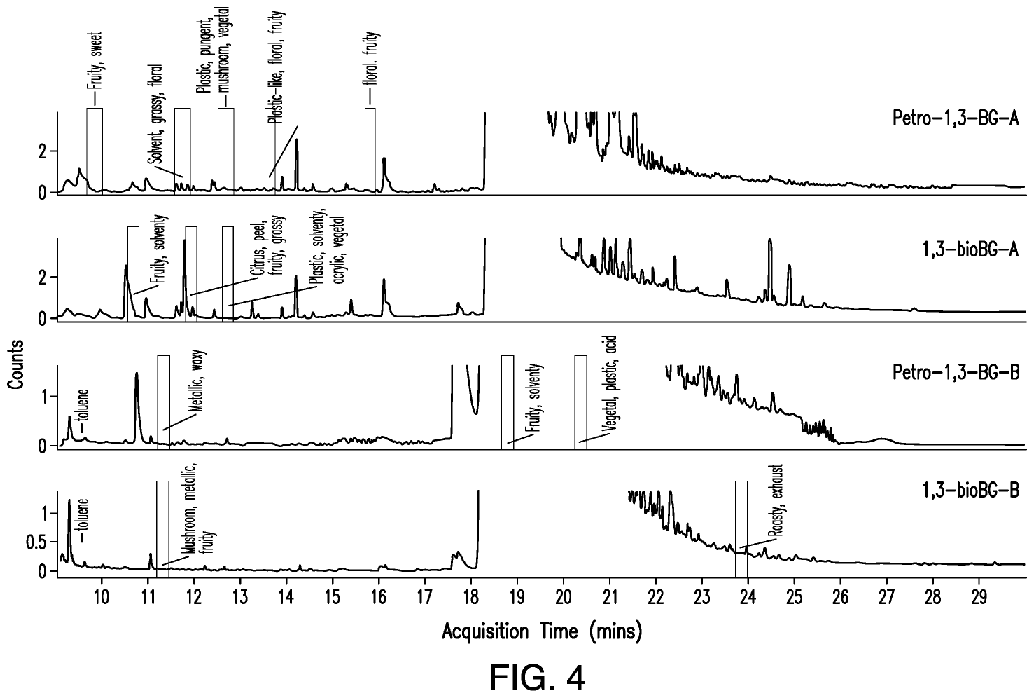

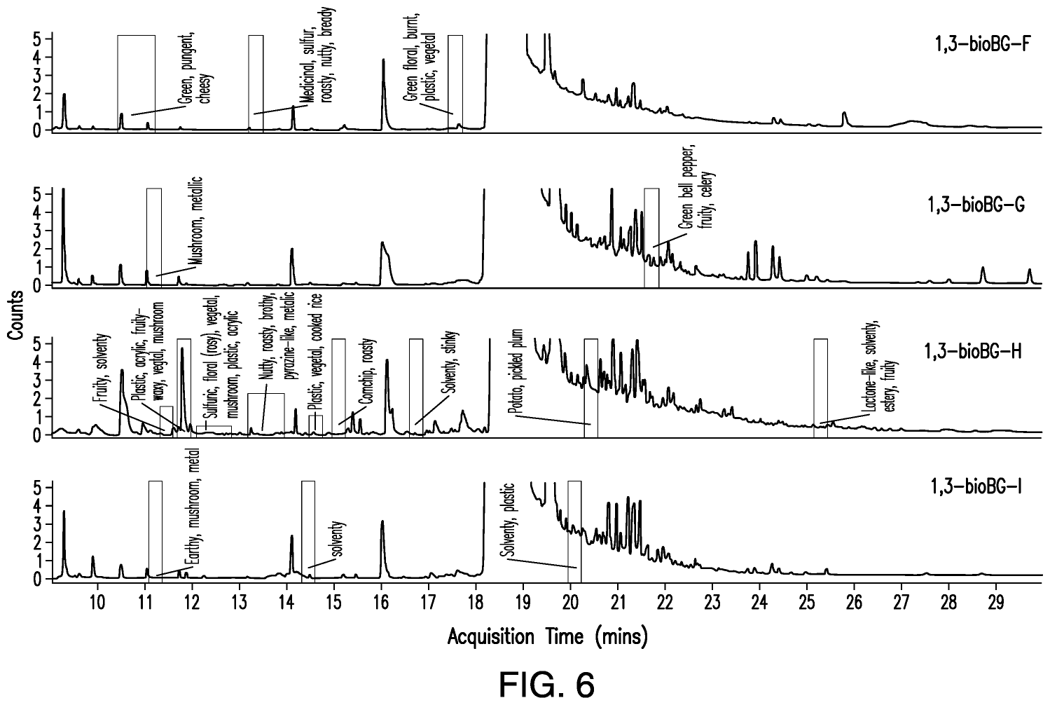

FIG. 4. to FIG. 6 show GC/MS chromatographs for 1,3-bioBG products and petro-1,3-BG samples with gas chromatography/olfactory (GC-O) analysis. Table 13 provides the GC-O peaks and their corresponding odors. The peaks are referred to as odor veins (also referred to herein as odor-active regions). As shown in FIGS. 3-6, the pure 1,3-BG chemical peak on the chromatograph is visible at about 18 mins-22 mins acquisition time which is a relative time of 1.0 for all samples.

TABLE 13
1,3-BG Odor-Active Regions.

| # | Sample | Acquisition Time/Relative Time | Odor |
|---|---|---|---|
| 1 | 1,3-bioBG-A | 10.7/0.594 | fruity, solventy |
| | | 11.8/0.655 | citrus, peel, fruity, grassy |
| | | 12.8/0.711 | plastic, solventy, acrylic, vegetal |
| 2 | 1,3-bioBG-B | 11.3/0.628 | mushroom, metallic, fruity |
| | | 23.9/1.328 | roasty, exhaust |
| 3 | 1,3-bioBG-C | 11.3/0.628 | mushroom, metallic, earthy, vegetal |
| | | 12.8/0.711 | fruity, grape wine |
| | | 13.4/0.744 | cooked potato, roasty, medicinal |
| | | 13.8/0.767 | roasty, dough |
| | | 17.7/0.983 | cooked pasta, fried |
| 4 | 1,3-bioBG-D | 11.8/0.655 | floral, fruity |
| | | 12.8/0.711 | plastic, mushroom, earthy, vegetal |
| | | 14.7/0.817 | plastic, vegetal |
| 5 | 1,3-bioBG-E | 11.2/0.622 | brothy, metallic, green |
| 6 | 1,3-bioBG-F | 10.5/0.583 | green, pungent, cheesy |
| | | 10.6/0.589 | green, pungent, cheesy |
| | | 10.7/0.594 | green, pungent, cheesy |
| | | 10.8/0.60 | green, pungent, cheesy |
| | | 10.9/0.605 | green, pungent, cheesy |
| | | 13.3/0.739 | medicinal, sulfur, roasty, nutty, bready |
| | | 17.5/0.972 | Green floral, burnt plastic, vegetal |
| 7 | 1,3-bioBG-G | 11.1/0.616 | mushroom, metallic |
| | | 21.8/1.211 | green bell pepper, fruity, celery |
| 8 | 1,3-bioBG-H | 11.4/0.633 | solventy, fruity, mushroom, vegetal |
| | | 11.8/0.655 | plastic, fruity, waxy |
| | | 12.2/0.678 | sulfuric, floral (rosey), vegetal, |
| | | | mushroom, plastic, acrylic |
| | | 12.3/0.683 | sulfuric, floral (rosey), vegetal, |
| | | | mushroom, plastic, acrylic |
| | | 12.4/0.689 | sulfuric, floral (rosey), vegetal, |
| | | | mushroom, plastic, acrylic |
| | | 12.5/0.694 | sulfuric, floral (rosey), vegetal, |
| | | | mushroom, plastic, acrylic |
| | | 12.6/0.70 | sulfuric, floral (rosey), vegetal, |
| | | | mushroom, plastic, acrylic |
| | | 12.7/0.705 | sulfuric, floral (rosey), vegetal, |
| | | | mushroom, plastic, acrylic |
| | | 12.8/0.711 | sulfuric, floral (rosey), vegetal, |
| | | | mushroom, plastic, acrylic |
| | | 13.3/0.739 | nutty, roasty, brothy, pyrazine-like, |
| | | | metallic |
| | | 13.4/0.744 | nutty, roasty, brothy, pyrazine-like, |
| | | | metallic |
| | | 13.5/0.75 | nutty, roasty, brothy, pyrazine-like, |
| | | | metallic |
| | | 13.6/0.755 | nutty, roasty, brothy, pyrazine-like, |
| | | | metallic |
| | | 13.7/0.761 | nutty, roasty, brothy, pyrazine-like, |
| | | | metallic |
| | | 14.6/0.816 | plastic, vegetal, cooked rice |
| | | 15/0.833 | corn chip, roasty |
| | | 16.7/0.928 | solventy, stinky |
| | | 20.5/1.139 | potato, pickled plum |
| | | 25.3/1.405 | lactone-like, solventy, estery, fruity |
| 9 | 1,3-bioBG-I | 11.3/0.628 | earthy, mushroom, metal |
| | | 14.4/0.8 | solventy |
| | | 20.1/1.117 | solventy, plastic |
| 10 | petro-1,3-BG-A | 9.8/0.544 | fruity, sweet |
| | | 11.8/0.655 | solvent, grassy, floral |
| | | 12.8/0.71 | plastic, pungent, mushroom, vegetal |
| | | 13.8/0.767 | plastic-like, floral, fruity |
| | | 15.7/0.872 | floral, fruity |
| 11 | petro-1,3-BG-B | 11.3/0.628 | metallic, waxy |
| | | 18.3/1.017 | fruity, solventy |
| | | 20.5/1.139 | vegetal, plastic, acid |
| 12 | petro-1,3-BG-C | 10.8/0.60 | fruity |
| | | 11.4/0.633 | corn chip, plastic |
| | | 18.6/1.033 | solventy, fruity |

The GC/MS and GC-O analysis revealed that neutral odor 1,3,-bioBG-A had a decreased number of odor-active regions as compared with petro-1,3-BG industry standards (petro-1,3-BG-A, petro-1,3-BG-B, and petro-1,3-BG-C). Neutral odor 1,3-bioBG-B that was purified by multi-step distillation described in Example 2 had only two odor peaks at 0.628 (mushroom, metallic, fruity) and 1.328 (roasty, exhaust) relative time to pure 1,3-BG. This was also the case for 1,3-bioBG-G, which had two odor peaks at 0.616 (mushroom, metallic) and 1.211 (green bell pepper, fruity, celery) relative time to pure 1,3-BG.

### Example 5: Identification of Aroma Compounds in 1,3-bioBG

Aroma analysis of the samples in Tables 11 and Table 13, was performed. A sensory panel of 5 panelists with experience in sensory analysis evaluated the 1,3-bioBG test samples and petro-1,3,-BG controls to assess aroma attributes that differentiated the samples. The samples were diluted with water to 1% v/v and 4% v/v and presented to panelists in 4-ounce plastic souffle cups.

Volatile aroma extraction was performed. One-hundred milliliters of off-flavor samples or control samples were spiked with 5 μg of 2-methyl-3-heptanone as an internal standard and mixed with 100 mL of nano-filtered water in a separatory funnel. The sample was then extracted three times using a 90/10 mixture of hexane/acetone (1×60 mL and 2× 40 mL each). The organic layers were pooled and dried over sodium sulfate anhydrous and then concentrated to 1 mL using a BioTage™ TurboVap Evaporator (1.5 L/min, 55° C.) and finally concentrated to a final volume of 200 μL with nitrogen evaporation at room temperature (20° C.). The concentrated extract was kept stored at −80° C. until analysis. The final extracts exhibited the characteristic aroma of the samples when a drop of the solution was vaporized on a strip of filter paper.

The aroma attributes that were used by the panel to describe the off-flavor sample were urine-like, sweaty, and medicinal/antibiotic ointment. The aroma of the control sample was described as medicinal/antibiotic ointment and oily.

GC/MS/O analyses were performed as described in Examples 3-4. Five aroma regions were detected in total in the sample extracts by GC/MS/O analysis: four aroma regions were detected only in one off-flavor sample, while one was only detected in the control sample The aroma regions in the off-flavor sample were described by the two panelists as having solventy, plastic, musty, mushroom, earthy, sulfury, and urine-like aromas. In contrast, the aroma region in the control sample was described as fruity. Analysis of the sample extracts by GC/QTOF-MS enabled identification of the aroma compounds (Table 14) based on pure compound standard or accurate mass EI spectra, NIST17 library matches, and LRIs. The mushroom, earthy-smelling compound, 1-octen-3-one, was positively identified via injection of pure standard. Tentative identifications were made for the other three aroma compounds detected in the off-flavor sample and one aroma compound detected in the control sample. For the “off-flavor” sample, these compounds were identified as: 2-methoxymethyl-2,4,5-trimethyl-1,3-dioxolane (solventy), 3-heptanone, (plastic, musty), and N-pentyl-2-butylamine (sulfury, urine-like). In the control sample, 5-norbornene-2-ol (fruity) was also identified.

TABLE 14
Aroma Compound Identification.

| Sample | Odor Description² | Compound | Library MatchScore³ |
|---|---|---|---|
| Off-flavor | Solventy | 2-Methoxymethyl-2,4,5-trimethyl- | 80.88 |
| | | 1,3-dioxolane | |
| Control | Fruity | 5-Norbornene-2-ol | 82.42 |
| Off-flavor | Plastic, musty | 3-Heptanone | 94.05 |
| Off-flavor | Mushroom, earthy | 1-Octen-3-one | Confirmed with pure |
| | | | standard |
| Off-flavor | Sulfury, urine-like | N-Pentyl-2-butylamine | 70.98 |

¹ Experimental linear retention index on a DB-5 ms column.
²Odor description from GC/MS/O analysis of sample extract.
³Library match scores were calculated by Agilent Unknowns Analysis based on the NIST17 library and are on a 100-point scale.

The relative concentration of the identified aroma compounds in the off-flavor sample compared to the control sample were calculated using internal standard normalized peak areas and are reported in Table 15. For the four aroma compounds detected only in the off-flavor sample, the relative percent increase in compound concentration (peak area) compared to the control ranged from 124% to 279,628%. For the compound detected only in the control sample (tentatively identified as 5-norbornene-2-ol), the relative percent decrease in compound concentration in the off-flavor sample was 42%.

TABLE 15
Relative concentration of aroma compounds in a non-distilled
1,3-bioBG sample compared to 1,3-bioBG-B sample.

| | Odor | Relative Concentration Off- |
|---|---|---|
| Compound | Threshold(μg/L)¹ | Flavor vs. Control² |
| 2-Methoxymethyl-2,4,5- | NR | +538% |
| trimethyl-1,3-dioxolane | | |
| 5-Norbornene-2-ol | NR | −42% |
| 3-Heptanone³ | 80 | +29,560% |
| 1-Octen-3-one | 0.016 | +124% |
| N-Pentyl-2-butylamine³ | NR | +279,628% |

¹Odor threshold value in water; retrieved from van Gemert, L.J. (2011). Odour Thresholds 2^{nd} Edition. NR stands for not reported.
²Relative concentrations were calculated based on internal standard normalized peak areas.
³The noise was used as an estimate of the peak area of the compound in the control sample.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

1. A bioderived 1,3-butylene glycol (1,3-BG) composition, comprising:
(a) an odor-active region having a relative acquisition time of 1.328;
(b) a fruity and/or solventy odor-active region having a relative acquisition time of 0.594;
(c) a fruity odor-active region having a relative acquisition time of 0.628;
(d) a citrus and/or peel odor-active region having a relative acquisition time of 0.655;
(e) a solventy odor-active region having a relative acquisition time of 0.711; or
(f) an odor concentration at or lower than the perception threshold at one or more odor-active regions having a relative acquisition time selected from the group consisting of: 0.544, 0.583, 0.589, 0.594, 0.60, 0.605, 0.616, 0.622, 0.628, 0.633, 0.655, 0.678, 0.683, 0.689, 0.694, 0.70, 0.705, 0.711, 0.739, 0.744, 0.750, 0.755, 0.761, 0.767, 0.80, 0.816, 0.817, 0.833, 0.872, 0.928, 0.972, 0.983, 1.017, 1.033, 1.117, 1.139, 1.211 and 1.405,
wherein in GC-O analysis under specified conditions the relative acquisition time of 1,3-BG is 1.0.

2. The bioderived 1,3-BG composition of claim 1, wherein the odor-active region having a relative acquisition time of 1.328 comprises an odor selected from roast and/or exhaust.

3. The bioderived 1,3-BG composition of claim 1, further comprising (a) a fruity and/or solventy odor-active region having a relative acquisition time of 0.594; (b) a fruity odor-active region having a relative acquisition time of 0.628; (c) a citrus and/or peel odor-active region having a relative acquisition time of 0.655; or (d) a solventy odor-active region having a relative acquisition time of 0.711, wherein in GC-O analysis under specified conditions the relative acquisition time of 1,3-BG is 1.0.

4. The bioderived 1,3-BG composition of claim 1, wherein the odor in the odor-active region having a relative acquisition time of 1.328 is below the perception threshold.

5. The bioderived 1,3-BG composition of claim 1, wherein (a) the fruity and/or solventy odor-active region having a relative acquisition time of 0.594 is below the perception threshold; (b) the fruity odor-active region having a relative acquisition time of 0.628 is below the perception threshold; (c) the citrus and/or peel odor-active region having a relative acquisition time of 0.655 is below the perception threshold; or (d) the solventy odor-active region having a relative acquisition time of 0.711 is below the perception threshold.

6. (canceled)

7. The bioderived 1,3-BG composition of claim 1, wherein (a) the fruity and/or solventy odor-active region having a relative acquisition time of 0.594 has an odor concentration between at least about 1 o.u./m³ up to 10000 o.u./m³.

8. The bioderived 1,3-BG composition of claim 1, further comprising an odor-active region having a relative acquisition time of 1.328, wherein in GC-O analysis under specified conditions the relative acquisition time of 1,3-BG is 1.0.

9. The bioderived 1,3-BG composition of claim 8, wherein the odor-active region having a relative acquisition time of 1.328 comprises an odor selected from roasty and/or exhaust.

10. The bioderived 1,3-BG composition of claim 1, wherein (a) the fruity and/or solventy odor-active region having a relative acquisition time of 0.594 is below the perception threshold; (b) the fruity odor-active region having a relative acquisition time of 0.628 is below the perception threshold; (c) the citrus and/or peel odor-active region having a relative acquisition time of 0.655 is below the perception threshold; or (d) the solventy odor-active region having a relative acquisition time of 0.711 is below the perception threshold.

11. The bioderived 1,3-BG composition of claim 8, wherein the odor in the odor-active region having a relative acquisition time of 1.328 is below the perception threshold.

12. (canceled)

13. The bioderived 1,3-BG composition of claim 1, wherein the one or more odor-active regions contain an odor selected from the group consisting of fruity, sweet, green, pungent, cheesy, solventy, mushroom, metallic, brothy, waxy, earthy, vegetal, corn chip, plastic, grassy, floral, citrus, peel, sulfuric, floral (rosy), acrylic, pungent, grape wine, medicinal, roasty, nutty, bready, cooked potato, pyrazine-like, plastic-like, dough, cooked rice, stinky, green floral, burnt plastic, cooked pasta, fried, acid, potato, pickled plum, green bell pepper, celery, exhaust, lactone-like and estery.

14. The bioderived 1,3-BG composition of claim 1, further comprising an odor concentration at or lower than the perception threshold at an odor-active region having a relative acquisition time of 1.328 and having an odor selected from roasty and/or exhaust.

15. The bioderived 1,3-BG composition of claim 1, further comprising an odor concentration at or lower than the perception threshold at an odor-active region having a relative acquisition time of 0.628 and having a fruity odor.

16. The bioderived 1,3-BG composition of claim 1, wherein the one or more odor-active regions comprises a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30 31, 32, 33, 34, 35, 36 or 37 odor-active regions.

17. The bioderived 1,3-BG composition of claim 1, wherein the odor concentration at the one or more odor-active regions is lower than the perception threshold.

18. The bioderived 1,3-BG composition of claim 17, wherein (a) the odor concentration at the one or more odor-active regions is at least 50% lower than the perception threshold; or (b) the odor concentration at the one or more odor-active regions is between 55% and 100% lower than the perception threshold.

19. (canceled)

20. A composition, comprising the bioderived 1,3-butylene glycol (1,3-BG) of claim 1; and a product ingredient, wherein the product ingredient is a cosmetic, fragrance, a personal care product, a flavoring substance, a food additive or a drink additive.

21. The composition of claim 20, wherein the bioderived 1,3-BG is included as a humectant, a solvent, a viscosity controlling agent, a diluent, a fragrance enhancer, or a flavor enhancer.

22. (canceled)
